(19) United States
(12) Patent Application Publication
(10) Pub. No.: US 2010/0048467 A1
(43) Pub. Date: Feb. 25, 2010

(54) Cystine-Knot Fold Cytokine

(75) Inventors: Mark Douglas Davies, London (GB); Christopher Benjamin Phelps, London (GB); Richard Joseph Fagan, London (GB); Christine Power, Thoiry (FR); Melanie Yorke, Bernex (CH); Mark Ibberson, Palezieux (CH); Yan Lavrovsky, Hull, MA (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: ARES TRADING S.A., Aubonne (CH)

(21) Appl. No.: 12/159,541

(22) PCT Filed: Jan. 12, 2006
(86) PCT No.: PCT/US2006/000951

§ 371 Date: Feb. 20, 2009

(51) Int. Cl. A61K 38/16; A61K 31/7088; A61P 35/00

(52) U.S. Cl. 514/12; 514/44 R

(57) ABSTRACT

This invention relates to a novel protein (INSP002), herein identified as a secreted protein that is a member of the Dan family of the cystine-knot fold cytokine superfamily and to the use of this protein and nucleic acid sequences from the encoding genes in the diagnosis, prevention and treatment of disease.

For purposes of the United States, this application is a continuation-in-part of U.S. application Ser. No. 10/872,898, filed Jun. 21, 2004, as a continuation-in-part of PCT/GB02/05865, filed Dec. 20, 2002, designating the U.S., and published as WO 03/055911 A2 on Jul. 10, 2003, and claimed priority from a GB application 0130738.8 filed 21 Dec. 2001, all of which is hereby incorporated herein by reference.

### BACKGROUND

This invention relates to the INSP002 protein, herein identified as a secreted protein that is a member of the Dan family of the cystine-knot fold cytokine superfamily and to the use of this protein and nucleic acid sequences from the encoding genes in the diagnosis, prevention and treatment of disease.

All publications, patents and patent applications cited herein are incorporated in full by reference.

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Secreted Protein Background

The ability for cells to make and secrete extracellular proteins is central to many biological processes. Enzymes, growth factors, extracellular matrix proteins and signalling molecules are all secreted by cells through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

Growth factors represent a relatively large group of polypeptides which share the property of inducing cell multiplication both in vivo and in vitro. Growth factors differ from classical endocrine hormones such as insulin or growth hormone in two important ways. Firstly, endocrine hormones are typically synthesised in specialised glands (such as the pancreas, in the case of insulin) whereas growth factors are often synthesised in multiple types of cells and tissues. Secondly, classical endocrine hormones are released into body fluids at the site of synthesis and are carried to their target tissue in the bloodstream. A hallmark of growth factors is that, in most instances, they act locally within the tissues that they are synthesised in (reviewed in Heath, J K. (1993) Growth Factors, Oxford University Press, Oxford, UK, pp. 15-33).

Although the level of sequence similarity between growth factors is not high, they can be classified into superfamilies based on their structural and functional similarities. Examples of these superfamilies include: (a) the hematopoietic growth factors, such as growth hormone, IL-2, IL-4, G-CSF, and CNTF, which all posses a four-helix-bundle structural motif; (b) the beta-trefoil family members, such as IL-1 beta, IL-1 alpha, FGF, and keratinocyte growth factor; (c) the EGF-like growth factors such as EGF and TGF alpha, which all have a immunoglobulin-like domain; and (d) the cystine-knot growth-factor fold which includes NGF, TGF beta, PDGF, and glycoprotein hormones.

Growth factors are extracellular and in order to exert a biological effect, they interact with specific, high affinity receptors located on the plasma membranes of target cells. The molecular characterisation of a variety of different growth factor receptors has revealed that they fall into defined families: the tyrosine kinase receptors, G-protein associated seven transmembrane receptors, and the serine/threonine kinase receptors. The tyrosine kinase receptors are characterised by an extracellular domain, a transmembrane domain, and an intracellular domain which possess tyrosine kinase activity. The serine/threonine kinase growth factor receptors are similar to to the tyrosine kinase receptors with an extracellular domain, a transmembrane domain, and an intracellular domain. The intracellular domain has intrinsic serine/threonine kinase activity.

Deregulation of growth factors is implicated in a variety of disease states, including, but not limited to, oncological diseases (Bartucci M et al, (2001) Cancer Res. September 15; 61(18):6747-54, Dias S et al., (2001) Proc Natl Acad Sci USA. September 11; 98(19):10857-62, Djavan B et al., (2001) World J Urol. 19(4):225-33), inflammatory diseases (Fiocchi C. (2001) J Clin Invest. August; 108(4):523-6, Hodge S et al., (2001) Respirology. September; 6 (3):205-211, Fenwick S A et al., (2001) J Anat. September; 199(Pt 3):231-40), neurological diseases (Cooper J D et al., (2001) Proc Natl Acad Sci USA 98(18):10439-44, Fahnestock M et al, (2001) Mol Cell Neurosci 18(2):210-20), and metabolic diseases (Vickers M H et al., (2001) Endocrinology. 142(9):3964-73).

Cystine Knot Fold Superfamily

The typical structure seen in the cystine knot superfamily is based on the presence of 6 cysteine residues creating 3 disulphide bonds. Two of the disulphide bonds create a ‘ring-like’ structure, which is penetrated by the third disulphide bond, (Sun et al. 1995). Cystine knot domains are often found with more than 6 cysteine residues. The extra cysteine residues are normally used to create further disulphide bonds within the cystine knot domain or interchain disulphide bonds, during dimerisation.

This cystine knot superfamily is divided into subfamilies, which include, the glycoprotein hormones (eg. follicle stimulating hormone), the transforming growth factor beta (TGFBeta) proteins (eg. bone morphogenetic protein 4), the platelet-derived growth factor-like (PDGF-like) proteins (eg. platelet derived growth factor A), nerve growth factors (NGF) (eg. brain-derived neurotrophic factor) and the differential screening-selected gene aberrative in neuroblastoma (DAN) family (eg. cerberus). The DAN subfamily includes Cerl, Cerberus, Caronte, Drm/Gremlin, PRDC, DAN, Dante and CeCan1 (Massague et al. Genes Dev 2000 Mar. 15; 14(6):627-44; Massague & Wotton, EMBO J. 2000 Apr. 17; 19(8):1745-54).

It is thought that members of the DAN subfamily may be able to modulate the actions of members of the TGFbeta subfamily of proteins (Pearce et al., Dev Biol. 1999 May 1; 209(1):98-110). More specifically, it is possible that members of the DAN subfamily are able to modulate the actions of bone morphogenetic proteins (BMPs) during development.

Members of the DAN subfamily have been found to act as antagonists of bone morphogenetic proteins (BMP), which are members of the TGFBeta subfamily of the cystine knot supefamily (Stanley et al., Mech Dev. 1998 October; 77(2):173-84; Massague et al. 2000 (supra); Massague J & Wotton D, 2002 (supra)). BMP monomers homo- or heterodimerise, through the binding of their cystine knot domains, before they interact with cell surface receptors. It is thought that DAN subfamily members are able to bind BMPs through their own cystine knot domains. This prevents the BMP from binding to its natural dimerisation partner and as a result, the BMP is no longer able to interact with its cell surface signaling receptor. Experiments specifically looking at DAN, Cerl, and DRM, have shown that they inhibit the action of BMP4 (Pearce et al. 1999, (supra)).

A greater understanding of the function of cerberus has been achieved as a result of binding studies (Piccolo S. et al., Nature. 1999 Feb. 25; 397(6721):707-10). The first functional studies carried out on cerberus used the Xenopus laevis cerberus protein (cer). Microinjection of Xeonpus cerberus mRNA in Xenopus embryos revealed that the cer protein induced formation of ectopic heads in the anterior endoderm of the Spemann's organizer (Bouwmeester et al. Nature 1996 Aug. 15; 382(6592):595-601; Bouwmeester T., Int J Dev Biol. 200, 145(1 Spec No):251-8). Binding studies carried out by Piccolo and co-workers revealed that the Xenopus cerberus protein binds and inhibit the actions of Nodal, BMP and Wnt proteins via independent sites. More specifically, they found that cerberus has a high specific affinity for and inhibitory effect on Xnr-1 (Nodal family member), BMP4 (BMP family member) and Xwmt-8 (Wnt family member). This work links cerberus, and hence other members of the DAN family, to developmental and tissue differentiation pathways.

Sclerostin, encoded by the gene SOST, is also a member of the DAN subfamily (Brumkow et al, 2001, Am. J. Hum. Genet. 68:577-589). SOST has been linked to sclerosteosis, an autosomal recessive sclerosing bone dysplasia. The phenotype associated by sclerosteosis is progressive skeletal overgrowth, which can lead to gigantism, distortion of the facies and entrapment of the seventh and eighth cranial nerves (Brumkow et al. 2001, (supra)). The link between sclerosteosis and SOST was determined through homozygosity mapping in families who are affected by the disease. Brumkow and co-workers identified a similarity between the phenotype associated with sclerosteosis and effects associated with other DAN subfamily members. This link was strengthened by the suggestion that sclerosteosis may arise due to lose of a negative regulator of TGFbeta subfamily member, more specially a BMP.

Identification of secreted proteins and in particular growth factors, such as members of the cystine knot fold superfamily, and in particular members of the DAN subfamily, is therefore of extreme importance in increasing understanding of the underlying pathways that lead to the disease states and associated disease states, mentioned above, and in developing more effective gene or drug therapies to treat these disorders.

The Invention

The invention is based on the discovery that the INSP002 protein functions as a secreted protein and moreover as a secreted protein of the DAN subfamily of the cystine knot fold cytokine superfamily.

The sequence of the INSP002 protein and the identification of this protein as a member of the DAN subfamily of cystine knot fold cytokines was first disclosed WO03/055911 and was subsequently confirmed in Katoh M. et al (Oncol Rep. 2004 August; 12(2):423-7) and Marques et al. (Genes Dev. 2004 Oct. 1; 18(19):2342-7). The INSP002 polypeptide is designated CKTSF1B3 and Cerebus2, respectively, in these two journal publications. A spice variant of the INSP002 polypeptide, designated Coco, was confirmed as a member of the DAN subfamily of cystine knot fold cytokines in Bell et al. (Development. 2003 April; 130(7):1381-9).

Surprisingly, it has now been found that the INSP002 polypeptide to functions/acts to inhibit TGFbeta-induced production of IL-11 by cancer cells. Elevated TGFbeta expression is a feature of many advanced tumours and is known to promote production of IL-11, an osteolytic factor that promotes metastasis of tumours to the skeleton and causes bone destruction. The activity of TGFbeta in promoting metastasis via release of IL-11 has been well-documented in breast cancer (see, for example, Tang et al, 2003, J. Clin Investig, 112, 7: 1116). In view of the role of TGFbeta in cancer, attempts have been made to develop TGFbeta inhibitors, such as monoclonal antibodies against TGFbeta, for the treatment of cancer. The ability of the INSP002 polypeptide to reduce release of IL-11 from cancer cells by inhibiting TGFbeta strongly suggests that the INSP002 polypeptide is a promising therapeutic agent for the treatment of cancer. Furthermore, the data presented herein show that, at high concentrations, the INSP002 polypeptide inhibits proliferation of cancer cells and even kills the cancer cells.

The ability of the INSP002 polypeptide to act as a TGFbeta antagonist and decrease IL-11 levels suggests that it may be useful in the treatment of cancer, in particular metastatic cancer and metastatic breast cancer, and in the treatment of other oncological disorders such as bone marrow failure and acute myeloid leukaemia. The finding that the INSP002 polypeptide decreases IL-11 levels also suggests that antagonists of the INSP002 polypeptide may be useful in the treatment or diagnosis of diseases in which low levels of IL-11 are implicated, such as autoimmune diseases and shock.

In a first aspect, the invention provides a method of treating cancer or a disorder associated with cancer comprising administering to a patient in need thereof a polypeptide which:
(i) comprises or consists of the amino acid sequence as recited in SEQ ID NO:2, SEQ ID NO:4, SEQ ID NO: 7, SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27;
(ii) is a fragment thereof having the function of a secreted protein, preferably the function of a member of the cystine knot fold cytokine superfamily, preferably a member of the DAN subfamily, or having an antigenic determinant in common with the polypeptides of (i); or
(iii) is a functional equivalent of (i) or (ii).

According to a further embodiment of the first aspect of the invention, there is provided a polypeptide as described above for use in the treatment of cancer or a disorder associated with cancer. The invention also provides the use of a polypeptide as described above in the manufacture of a medicament for the treatment of cancer or a disorder associated with cancer.

Preferably, the cancer is metastatic cancer. By “metastatic cancer” is meant that the cancer has spread from the site of the primary tumour. The cancer or metastatic cancer that is treated acording to the methods and uses of the invention may be any form of cancer including, but not limited to, cancers of the brain, blood (such as acute myeloid leukaemia), skin, liver, kidney, breast, colon, and lung. Preferably, the cancer to be treated is breast cancer, preferably metastatic breast cancer. Diseases associated with cancer that may be treated include bone marrow failure.

According to a further embodiment of the first aspect of the invention, the cancer is preferably treated by inhibiting the activity of TGFbeta. Preferably, inhibiting the activity of TGFbeta results in a reduction in the levels of IL-11 released from cancer cells. The invention therefore provides a method of treating cancer or a disorder associated with cancer by reducing levels of TGFbeta-induced IL-11 comprising administering to a patient in need thereof a polypeptide which:
(i) comprises or consists of the amino acid sequence as recited in SEQ ID NO:2, SEQ ID NO:4, SEQ ID NO: 7, SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27;
(ii) is a fragment thereof having the function of a secreted protein, preferably the function of a member of the cystine knot fold cytokine superfamily, preferably a member of the DAN subfamily, or having an antigenic determinant in common with the polypeptides of (i); or
(iii) is a functional equivalent of (i) or (ii).

There is also provided the use of a polypeptide as described above in the manufacture of a medicament for the treatment of cancer, wherein said treatment comprises reducing levels of TGFbeta-induced IL-11.

By “reducing levels of TGFbeta-induced IL-11” is meant that the levels of IL-11 produced by tumour cells in the presence of the polypeptide and TGFbeta are lower that the levels of IL-11 produced in the presence of TGFbeta alone. Preferably, levels of TGFbeta-induced IL-11 are reduced by at least 20% in the presence of TGFbeta and the polypeptide compared with levels in the presence of TGFbeta alone. Preferably, levels of TGFbeta-induced IL-11 are reduced by 30%, 40%, 50%, 60%, 70%, 80%, 90% or more, preferably by 95%, 96%, 97%, 98% or 99%. Preferably, levels IL-11 produced by the tumour cells are reduced in the presence of the polypeptide so that they are substantially the same as levels of IL-11 produced in the absence of TGFbeta. Reduction of TGFbeta-induced IL-11 levels in the tumour cells can be measured using the assays described herein in which levels of IL-11 produced by tumour cells incubated with TGFbeta alone are compared to levels of IL-11 produced by tumour cells incubated with TGFbeta and the polypeptide.

According to a further embodiment of the first aspect of the invention, the method preferably inhibits proliferation of the tumour cells. The invention therefore provides a method of treating cancer or a disorder associated with cancer by inhibiting proliferation of tumour cells comprising administering to a patient in need thereof a polypeptide which:
(i) comprises or consists of the amino acid sequence as recited in SEQ ID NO:2, SEQ ID NO:4, SEQ ID NO: 7, SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27;
(ii) is a fragment thereof having the function of a secreted protein, preferably the function of a member of the cystine knot fold cytokine superfamily, preferably a member of the DAN subfamily, or having an antigenic determinant in common with the polypeptides of (i); or
(iii) is a functional equivalent of (i) or (ii).

There is also provided the use of a polypeptide as described above in the manufacture of a medicament for the treatment of cancer, wherein said treatment comprises inhibiting proliferation of tumour cells.

By “inhibits proliferation of tumour cells” is meant that the rate of growth of the tumour cells is slower in the presence of the polpeptide compared to in the absence of the polypeptide. Inhibition of proliferation of tumour cells can be assessed by comparing the increase in the number of tumour cells in vitro over a period of time in the presence and absence of the polypeptide. Preferably, proliferation of tumour cells is inhibited by 20% in the presence of the polypeptide compared to in the absence of the polypeptide. Preferably, proliferation of tumour cells is inhibited by 30%, 40%, 50%, 60%, 70%, 80%, 90% or more, preferably by 95%, 96%, 97%, 98% ot 99%. Preferably, proliferation of the tumour cells is inhibited by approximately 100% such that there is no significant alteration in the number of tumour cells following addition of the polypeptide.

Preferably, inhibition of proliferation results from the polypeptide killing the tumour cells. By “killing tumour cells” is meant that there is a decrease in the number of tumour cells in the presence of the polypeptide, taking proliferation of the remaining cells into account.

Preferably, killing of tumour cells results, not only in inhibition of proliferation but also a decrease in the number or tumour cells. Preferably, the number of tumour cells is reduced by 20% in the presence of the polypeptide compared to the number of tumour cells originally present. Preferably, cell killing reduces the number of tumour cells by 30%, 40%, 50%, 60%, 70%, 80%, 90% or more, preferably by 95%, 96%, 97%, 98% ot 99%. Preferably, the number of tumour cells is reduced by approximately 100% such that there are substantially no tumour cells remaining following addition of the polypeptide.

The data presented herein show that the polypeptide inhibits TGFbeta-induced IL-11 release from tumour cells at low concentrations and is capable of inhibiting cell proliferation and even killing tumour cells at higher concentrations. It is within the ability of the skilled person to determiine the optimum dose of polypeptide that should be used to order to achieve the desired effect. Preferably, the methods and uses of the first aspect of the invention employ the polypeptide at a concentration of between 0.01 and 1000 ng/ml, preferably between 0.1 and 750 ng/ml, preferably between 1 and 500 ng/ml, preferably between 1 and 250 ng/ml, preferably between 1 and 100 ng/ml, preferably between 3 and 80 ng/ml, preferably between 5 and 50 ng/ml, preferably between 8 and 40 ng/ml.

The polypeptide having the sequence recited in SEQ ID NO:2 is referred to hereafter as “the INSP002 exon 1 polypeptide”. The polypeptide having the sequence recited in SEQ ID NO:4 is referred to hereafter as “the INSP002 exon 2 polypeptide”. The polypeptide having the sequence recited in SEQ ID NO:6 is produced by combining SEQ ID NO:2 and SEQ ID NO:4 and is referred to hereafter as “the INSP002 polypeptide”.

The first 22 amino acids of the INSP002 exon 1 polypeptide are a signal peptide and the INSP002 polypeptide sequences without the signal sequence are recited in SEQ ID NO: 7 and SEQ ID NO:8. The polypeptide having the sequence recited in SEQ ID NO:7 is referred to hereafter as “the INSP002 exon 1 polypeptide without signal peptide”. The polypeptide having the sequence recited in SEQ ID NO:8 is produced by combining SEQ ID NO:7 and SEQ ID NO:4 and is referred to hereafter as “the INSP002 polypeptide without signal peptide”.

The polypeptide having the sequence recited in SEQ ID NO:14 is a variant of the INSP002 polypeptide. It is identical to the INSP002 polypeptide except that it contains a two amino acid deletion at positions 107 and 108 and a single amino acid substitution at position 110 compared to the INSP002 polypeptide. The polypeptide having the sequence recited in SEQ ID NO:14 is referred to hereafter as “the variant INSP002 polypeptide”. The first 22 amino acids of the variant INSP002 polypeptide are a signal peptide and the invention also includes the INSP002 polypeptide without this signal peptide.

The INSP002 polypeptides described above or fragments thereof used in the invention may form part of a fusion protein. The sequences of exemplary fusions proteins included in the invention are provided above. The polypeptide having the sequence recited in SEQ ID NO:17 is a fusion protein of the INSP002 polypeptide without signal peptide and an Fc domain. The polypeptide having the amino acid sequence recited in SEQ ID NO:19 is a fusion protein of the N-terminal fragment of the INSP002 polypeptide without signal peptide (the first 71 amino acids) and an Fc domain. The polypeptide having the amino acid sequence recited in SEQ ID NO:21 is a fusion protein of the C-terminal fragment of the INSP002 polypeptide without signal peptide (amino acid 72 onwards) and an Fc domain. The polypeptide having the amino acid sequence recited in SEQ ID NO:23 is a fusion protein of the N-terminal fragment of the INSP002 polypeptide without signal peptide (the first 71 amino acids) and the alpha subunit of human chorionic gonadotrophin hormone (hCG) having a deletion of amino acids 88-92 (alpha des88-92). The polypeptide having the amino acid sequence recited in SEQ ID NO:25 is a fusion protein of the N-terminal fragment of the INSP002 polypeptide without signal peptide (the first 71 amino acids) and the beta subunit of hCG. The polypeptide having the amino acid sequence recited in SEQ ID NO: 27 is the INSP002 polypeptide without signal peptide with a tag of 6 histidine residues at the N-terminal.

The fusion proteins described above may form homodimers and heterodimers and these are also included in the invention. For example, the fusion protein having the amino acid sequence of SEQ ID NO:23 containing the hCG alpha subunit will interact with the fusion protein having the amino acid sequence of SEQ ID NO:25 containing the hCG beta subunit to form a hybrid heterodimeric fusion protein. Further details of alternative fusion proteins and hybrid multimeric fusion proteins that may be employed in the invention are provided below. The fusion proteins having the amino acid sequences of SEQ ID NO:17, SEQ ID NO:19, SEQ ID NO:21, SEQ ID NO:23 and SEQ ID NO:25, SEQ ID NO:27, hybrid multimeric fusion proteins comprising more than one of these specific amino acid sequences, and the additional fusion proteins described below are referred to herein as the “INSP002 fusion polypeptides”.

The term “INSP002 polypeptides” as used herein includes polypeptides comprising the INSP002 exon 1 polypeptide, the INSP002 exon 1 polypeptide without signal peptide, the INSP002 exon 2 polypeptide, the INSP002 polypeptide or the INSP002 polypeptide without signal peptide, as well as polypeptides consisting of the INSP002 exon 1 polypeptide, the INSP002 exon 1 polypeptide without signal peptide, the INSP002 exon 2 polypeptide, the INSP002 polypeptide, the INSP002 polypeptide without signal peptide, the variant INSP002 polypeptide and the INSP002 fusion polypeptides. The term “INSP002 polypeptides” includes these proteins in monomeric form or in dimeric form.

Preferably, the “member of the cystine knot fold cytokine family” included in the first aspect of the invention may be a molecule containing a cystine knot fold cytokine domain, preferably a DAN subfamily domain, detected with an e-value lower than 0.1, 0.01, 0.001, 0.0001, 0.00001, 0.000001 or 0.0000001.

Preferably, an INSP002 polypeptide functions as a member of the cystine knot fold cytokine superfamily, preferably as a member of the DAN subfamily. By “functions as a member of the cystine knot fold superfamily”, we refer to polypeptides that comprise amino acid sequence or strctural features that can be identified as conserved features within a cystine knot fold family protein, such that a biological activity is shared with a cystine knot fold family member. The term “cystine knot fold cytokine” is well understood in the art and the skilled worker will readily be able to ascertain whether a polypeptide functions as a member of the cystine knot fold cytokine superfamily using one of a variety of assays known in the art.

In particular, the skilled person may be able to ascertain whether a polypeptide functions as a member of the DAN subfamily by assaying whether it is an antagonist of TGF-beta superfamily members and whether it is a BMP antagonist.

The Xenopus embryo may be used as a system for assaying whether a polypeptide functions as a BMP antagonist since several BMPs are expressed in the Xenopus embryo (Chang C. et al. 1999, Development 126:3347-3357, Hawley S. et al., 1995, Genes Dev. 9:2923-2935, Hemmati-Brivanlou, A., and G. H. Thomsen. 1995, Dev. Genet. 17:78-89, Jones C. M. et al., 1992, Development 115:639-647). Overexpression of BMP-2/4-class or BMP-7-class signals in the early mesoderm induces ventral fates, while inhibitors of these signals (such as Noggin, Xnr3, Chordin, or Follistatin) induce dorsal fates. The effect of a polypeptide on embryonic development can therefore be used to determine whether that polypeptide is a BMP antagonist.

The ability of a polypeptide to act as a TGFbeta antagonist may be determined by assessing the ability of the polypeptide to inhibit the activities known to be associated with TGFbeta. Preferably, the polypeptide of the invention acts to reduce the release of IL-11 by cancer cells induced by TGFbeta. The ability of a polypeptide to reduce TGF-beta-induced release of IL-11 may be assessed by a cell-based assay such those described in the examples herein. Briefly, cancer cells are incubated with TGFbeta in the presence or absence of the polypeptide and, after incubation, levels of IL-11 are measured using a standard ELISA kit. Preferably, levels of TGFbeta-induced IL-11 are reduced by at least 20% in the presence of TGFbeta and the polypeptide compared with levels in the presence of TGFbeta alone. Preferably, levels of TGFbeta-induced IL-11 are reduced by 30%, 40%, 50%, 60%, 70%, 80%, 90% or more, preferably by 95%, 96%, 97%, 98% or 99%.

An “antigenic determinant” of the present invention may be a part of a polypeptide of the present invention, which binds to an antibody-combining site or to a T-cell receptor (TCR). Alternatively, an “antigenic determinant” may be a site on the surface of a polypeptide of the present invention to which a single antibody molecule binds. Generally an antigen has several or many different antigenic determinants and reacts with antibodies of many different specificities. Preferably, the antibody is immunospecific to a polypeptide of the invention. Preferably, the antibody is immunospecific to a polypeptide of the invention, which is not part of a fusion protein. Preferably, the antibody is immunospecific to the INSP002 polypeptide or a fragment thereof. Antigenic determinants usually consist of chemically active surface groupings of molecules, such as amino acids or sugar side chains, and can have specific three dimensional structural characteristics, as well as specific charge characteristics. Preferably, the “antigenic determinant” refers to a particular chemical group on a polypeptide of the present invention that is antigenic, ie. that elicit a specific immune response.

In a second aspect, the invention provides that the method or use of the first aspect of the invention comprises administering a purified nucleic acid molecule which encodes a polypeptide as described above.

The term “purified nucleic acid molecule” preferably refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “purified nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. In a preferred embodiment, genomic DNA are specifically excluded from the scope of the invention. Preferably, genomic DNA larger than 10 kbp (kilo baise pairs), 50 kbp, 100 kbp, 150 kbp, 200 kbp, 250 kbp or 300 kbp are specifically excluded from the scope of the invention. Preferably, the “purified nucleic acid molecule” consists of cDNA only.

Preferably, the purified nucleic acid molecule comprises the nucleic acid sequence as recited in SEQ ID NO:1 (encoding the INSP002 exon 1 polypeptide), SEQ ID NO:3 (encoding the INSP002 exon 2 polypeptide), SEQ ID NO:5 (encoding the INSP002 polypeptide), SEQ ID NO:13 (encoding the variant INSP002 polypeptide), SEQ ID NO:16, SEQ ID NO:18, SEQ ID NO:20, SEQ ID NO:22, SEQ ID NO:24 or SEQ ID NO:26 (encoding the INSP002 fusion polypeptides) or is a redundant equivalent or fragment of these sequences.

The invention further provides that the purified nucleic acid molecule consists of the nucleic acid sequence as recited in SEQ ID NO:1 (encoding the INSP002 exon 1 polypeptide), SEQ ID NO:3 (encoding the INSP002 exon 2 polypeptide), SEQ ID NO:5 (encoding the INSP002 polypeptide) or SEQ ID NO:13 (encoding the variant INSP002 polypeptide), SEQ ID NO:16, SEQ ID NO:18, SEQ ID NO:20, SEQ ID NO:22, SEQ ID NO:24 or SEQ ID NO:26 (encoding the INSP002 fusion polypeptides) or is a redundant equivalent or fragment of either of these sequences.

According to one embodiment of this aspect of the invention, the purified nucleic acid molecule does not contain the 5′ untranslated region located upstream of the nucleic acid sequence encoding the INSP002 exon 1 polypeptide and the nucleic acid sequence encoding the INSP002 polypeptide (nucleotides 1 to 151 of SEQ ID NO:1 and SEQ ID NO:5). According to this embodiment, the purified nucleic acid molecule preferably comprises nucleotides 152 to 475 of SEQ ID NO:1 or nucleotides 152 to 721 of SEQ ID NO:5. The invention further provides a purified nucleic acid molecule consisting of nucleotides 152 to 475 of SEQ ID NO:1 or nucleotides 152 to 721 of SEQ ID NO:5. The nucleotide sequence coding for the INSP002 polypeptide without the 5′ untranslated region (nucleotides 152 to 721 of SEQ ID NO:5) is given in SEQ ID NO:11 and the nucleotide sequence coding for the INSP002 exon 1 polypeptide (nucleotides 152 to 475 of SEQ ID NO:1) without the 5′ untranslated region is given in SEQ ID NO:12.

According to a further embodiment of this aspect, the purified nucleic acid molecule does not encode the signal peptide located at the start of the INSP002 exon 1 polypeptide and the INSP002 polypeptide (nucleotides 152 to 217 of SEQ ID NO:1 and SEQ ID NO:5). According to this embodiment, the purified nucleic acid molecule preferably comprises nucleotides 218 to 475 of SEQ ID NO:1 (encoding the INSP002 exon 1 polypeptide without signal peptide) or nucleotides 218 to 721 of SEQ ID NO:5 (encoding the INSP002 polypeptide without signal peptide). The invention further provides a purified nucleic acid molecule consisting of nucleotides 218 to 475 of SEQ ID NO:1 (encoding the INSP002 exon 1 polypeptide without signal peptide) or nucleotides 218 to 721 of SEQ ID NO:5 (encoding the INSP002 polypeptide without signal peptide). The nucleotide sequence encoding the mature INSP002 polypeptide (SEQ ID NO:7) is given in SEQ ID NO:9 and the nucleotide sequence encoding the mature INSP002 exon 1 polypeptide is given in SEQ ID NO:10.

According to a further embodiment of this aspect of the invention, the purified nucleic acid molecule does not contain the 5′ untranslated region located upstream of the nucleic acid sequence encoding the variant INSP002 polypeptide (nucleotides 1 to 68 of SEQ ID NO:13). According to this embodiment, the purified nucleic acid molecule preferably comprises or consists of nucleotides 69 to 719 of SEQ ID NO:13. The nucleotide sequence coding for the variant INSP002 polypeptide without the 5′untranslated region (nucleotides 69 to 719 of SEQ ID NO:13) is given in SEQ ID NO:15.

In a third aspect, the invention provides that the method or use of the first aspect of the invention comprises administering a vector comprising a purified nucleic acid molecule which encodes a polypeptide as described above.

In a fourth aspect, the invention further provides that the polypeptide or nucleic acid molecule used in the methods and uses of the first aspect of the invention may be administered in the form of a pharmaceutical composition further comprising a pharmaceutically acceptable carrier.

As used herein, “functional equivalent” refers to a protein or nucleic acid molecule that possesses functional or structural characteristics that are substantially similar to a polypeptide or nucleic acid molecule of the present invention. A functional equivalent of a protein may contain modifications depending on the necessity of such modifications for the performance of a specific function. The term “functional equivalent” is intended to include the fragments, mutants, hybrids, variants, analogs, and chemical derivatives of a molecule.

Preferably, the “functional equivalent” may be a protein or nucleic acid molecule that exhibits any one or more of the functional activities of the polypeptides of the present invention.

Preferably, the “functional equivalent” may be a protein or nucleic acid molecule that displays substantially similar activity compared with INSP002 or fragments thereof in a suitable assay for the measurement of biological activity or function. Preferably, the “functional equivalent” may be a protein or nucleic acid molecule that displays identical or higher activity compared with INSP002 or fragments thereof in a suitable assay for the measurement of biological activity or function. Preferably, the “functional equivalent” may be a protein or nucleic acid molecule that displays 50%, 60%, 70%, 80%, 90%, 95%, 98%, 99%, 100% or more activity compared with INSP002 or fragments thereof in a suitable assay for the measurement of biological activity or function.

Preferably, the “functional equivalent” may be a protein or polypeptide capable of exhibiting a substantially similar in vivo or in vitro activity as the polypeptides of the invention. Preferably, the “functional equivalent” may be a protein or polypeptide capable of interacting with other cellular or extracellular molecules in a manner substantially similar to the way in which the corresponding portion of the polypeptides of the invention would. For example, a “functional equivalent” would be able, in an immunoassay, to diminish the binding of an antibody to the corresponding peptide (ie., the peptide the amino acid sequence of which was modified to achieve the “functional equivalent”) of the polypeptide of the invention, or to the polypeptide of the invention itself, where the antibody was raised against the corresponding peptide of the polypeptide of the invention. An equimolar concentration of the functional equivalent will diminish the aforesaid binding of the corresponding peptide by at least about 5%, preferably between about 5% and 10%, more preferably between about 10% and 25%, even more preferably between about 25% and 50%, and most preferably between about 40% and 50%.

For example, functional equivalents can be fully functional or can lack function in one or more activities. Thus, in the present invention, variations can affect the function, for example, of ubiquitin binding, ubiquitin recognition, interaction with ubiquitinated substrate protein, such as binding or proteolysis, subunit interaction, particularly within the proteasome, activation or binding by ATP, developmental expression, temporal expression, tissue-specific expression, interacting with cellular components, such as transcriptional regulatory factors, and particularly trans-acting transcriptional regulatory factors, proteolytic cleavage of peptide bonds in polyubiquitin and peptide bonds between ubiquitin or polyubiquitin and substrate protein, and proteolytic cleavage of peptide bonds between ubiquitin or polyubiquitin and a peptide or amino acid.

As indicated above, the finding that the INSP002 polypeptide decreases IL-11 levels suggests that antagonists of the INSP002 polypeptides described above may increase IL-11 levels and may therefore be useful in the treatment or diagnosis of diseases such as autoimmune diseases or shock that are associated with low IL-11 levels.

According to a fifth aspect of the invention, there is provided a method of treating a disease associated with low IL-11 levels comprising administering to a patient in need thereof a compound that is an antagonist of the INSP002 polypeptide. There is also provided the use of a compound that is an antagonist of the INSP002 poylpeptide in the manufacture of a medicament for treating a disease associated with low IL-11 levels. Preferably, the disease to be treated is an autoimmune disease or shock.

By “antagonist” is meant that the compound decreases the level or activity of the INSP002 polypeptide. Preferably, the compound reduces the ability of the INSP002 to suppress IL-11 levels. The compound thus preferably results in an increase in IL-11 levels. Preferably, the compound resuts in an increase in TGFbeta-induced IL-11 levels. Preferably, levels of IL-11 are increased by at least 20% in the presence of the compound. Preferably, levels of IL-11 are increased by 30%, 40%, 50%, 60%, 70%, 80%, 90% or more, preferably by 95%, 96%, 97%, 98% or 99%.

Preferably, the compound is a ligand that binds to the INSP002 polypeptide. Preferably, the compound is an antibody that binds to the INSP002 polypeptide. Details of screening methods suitable for identifying and isolating antagonists of the INSP002 polypeptide, and in particular antibodies that bind to the INSP002 polypeptide, are provided herein.

In a sixth aspect, the invention provides a fusion polypeptide. In a first embodiment of the sixth aspect of the invention, the fusion polypeptide comprises:
a) a first polypeptide which (i) comprises or consists of the amino acid sequence as recited in SEQ ID NO:2, SEQ ID NO:4, SEQ ID NO: 7, SEQ ID NO:6, SEQ ID NO:8, SEQ ID NO: 14, (ii) is a fragment thereof having the function of a secreted protein, preferably the function of a member of the cystine knot fold cytokine superfamily, preferably a member of the DAN subfamily, or having an antigenic determinant in common with the polypeptides of (i); or (iii) is a functional equivalent of (i) or (ii); and
b) a second heterologous polypeptide selected from an Fc domain, an alpha domain or a beta domain of hCG, or a histidine tag.

Preferably, the fusion polypeptide of the sixth aspect of the invention comprises or consists of an INSP002 amino acid sequence as recited in SEQ ID NO:17, SEQ ID NO:19, SEQ ID NO:21, SEQ ID NO:23, SEQ ID NO:25 or SEQ ID NO:27).

According to a further embodiment of the sixth aspect of the invention, there is provided a hybrid fusion protein which is a multimeric protein comprising more than one fusion polypeptide according to the first embodiment of the sixth aspect of the invention described above. The hybrid fusion protein may contain two, three, four of more fusion polypeptides according to the first embodiment of the sixth aspect of the invention described above. Preferably, the hybrid fusion protein is a dimer comprising two fusion polypeptides as described above. Where the hybrid fusion protein is a dimer, it may be a homodimer or a heterodimer. Preferably, the hybrid fusion protein comprises or consists of more than one fusion polypeptides selected from the fusion polypeptides having an amino acid sequence as recited in SEQ ID NO:17, SEQ ID NO:19, SEQ ID NO:21, SEQ ID NO:23, SEQ ID NO:25 or SEQ ID NO:27. The invention thus includes homodimers comprising-two fusion polypeptides having an amino acid sequence as recited in SEQ ID NO:17, SEQ ID NO:19, SEQ ID NO:21, SEQ ID NO:23, SEQ ID NO:25 or SEQ ID NO:27. The invention further includes heterodimers comprising combinations of these sequences. Examples of hybrid fusion proteins according to this embodiment of the invention include a hybrid fusion protein comprising a first fusion polypeptide having the amino acid sequence as recited in SEQ ID NO:17 and a second fusion polypeptide having the amino acid sequence as recited in SEQ ID NO:19; a hybrid fusion protein comprising a first fusion polypeptide having the amino acid sequence as recited in SEQ ID NO:17 and a second fusion polypeptide having the amino acid sequence as recited in SEQ ID NO:21; a hybrid fusion protein comprising a first fusion polypeptide having the amino acid sequence as recited in SEQ ID NO:19 and a second fusion polypeptide having the amino acid sequence as recited in SEQ ID NO:21; and a hybrid fusion protein comprising a first fusion polypeptide having the amino acid sequence as recited in SEQ ID NO:23 and a second fusion polypeptide having the amino acid sequence as recited in SEQ ID NO:25;

In a seventh aspect, the invention provides a purified nucleic acid molecule which encodes one of these INSP002 fusion polypeptides. Preferably, the purified nucleic acid molecule comprises of consists of a nucleotide sequence as recited in SEQ ID NO:16, SEQ ID NO:16, SEQ ID NO:18, SEQ ID NO:20, SEQ ID NO:22, SEQ ID NO:24 or SEQ ID NO:26.

In a eighth aspect, the invention provides a purified nucleic acid molecule which hydridizes under high stringency conditions with a nucleic acid molecule of the seventh aspect of the invention. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C.

In ninth aspect, the invention provides a vector, such as an expression vector, that contains a nucleic acid molecule of the seventh or eighth aspect of the invention. In a tenth aspect, the invention provides a host cell transformed with a vector of the ninth aspect of the invention.

In a eleventh aspect, the invention provides a ligand which binds specifically to, and which preferably inhibits the cystine knot fold cytokine activity of an INSP002 fusion polypeptide of the sixth aspect of the invention. Preferably, the ligand inhibits the function of a polypeptide of the sixth aspect of the invention which is a—member of the DAN subfamily of cystine knot fold cytokines. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a twelth aspect, the invention provides a compound that is effective to regulate the activity of an INSP002 fusion polypeptide of the sixth aspect of the invention. A compound of the twelth aspect of the invention may either increase (agonise) or decrease (antagonise) the activity of the polypeptide. Such ligands and compounds may be identified using the assays and screening methods disclosed herein.

Another aspect of this invention resides in the use of an INSP002 fusion polypeptide as a target for the screening of candidate drug modulators. A further aspect of this invention resides in methods of screening of compounds for therapy of disorders, comprising determining the ability of a compound to bind a INSP002 fusion polypeptide. A further aspect of this invention resides in methods of screening of compounds for therapy of disorders, comprising testing for modulation of the activity of an INSP002 fusion polypeptide, or a fragment thereof.

In a thriteenth aspect, the invention provides for the use of an INSP002 fusion polypeptide of the sixth aspect of the invention as a secreted protein. Preferably, the invention provides for the use of a polypeptide of the sixth aspect of the invention as a cytokine, more preferably as a cystine knot fold cytokine and in particular as a member of the DAN subfamily of cystine knot fold cytokines.

In an fourteenth aspect, the invention provides a pharmaceutical composition comprising a polypeptide of the sixth aspect of the invention, or a nucleic acid molecule of the seventh or eighth aspect of the invention, or a vector of the ninth aspect of the invention, or a host cell of the tenth aspect of the invention, or a ligand of the eleventh aspect of the invention, or a compound of the twelth aspect of the invention, in conjunction with a pharmaceutically-acceptable carrier.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of the those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

In a further preferred embodiment, a polypeptide of the invention, that may comprise a sequence having at least 85% of homology with INSP002, is a fusion protein.

These fusion proteins can be obtained by cloning a polynucleotide encoding a polypeptide comprising a sequence having at least 85% of homology with INSP002 in frame to the coding sequences for a heterologous protein sequence.

The term “heterologous”, when used herein, is intended to designate any polypeptide other than a human INSP002 polypeptide.

Example of heterologous sequences, that can be comprised in the soluble fusion proteins either at N- or at C-terminus, are the following: extracellular domains of membrane-bound protein, immunoglobulin constant regions (Fc region), multimerization domains, domains of extracellular proteins, signal sequences, export sequences, or sequences allowing purification by affinity chromatography.

Many of these heterologous sequences are commercially available in expression plasmids since these sequences are commonly included in the fusion proteins in order to provide additional properties without significantly impairing the specific biological activity of the protein fused to them (Terpe K, Appl Microbiol Biotechnol, 60: 523-33, 2003). Examples of such additional properties are a longer lasting half-life in body fluids, the extracellular localization, or an easier purification procedure as allowed by the a stretch of Histidines forming the so-called “histidine tag” (Gentz et al., Proc Natl Acad Sci USA, 86: 821-4, 1989) or by the “HA” tag, an epitope derived from the influenza hemagglutinin protein (Wilson et al., Cell, 37: 767-78, 1994). If needed, the heterologous sequence can be eliinated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. For example, the protein used in the examples (INSP002) can be purified by means of a hexa-histidine peptide fused at the C-terminus of INSP002. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (half-life), increased specific activity, increased expression level, or the purification of the fusion protein is facilitated.

In a preferred embodiment, the protein is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM or IgA, for example. Fusion proteins may be monomeric or multimeric, hetero- or homomultimeric.

Non-immunoglobulin proteins such as heterodimeric proteinaceous hormones, can also be used for making fusion proteins. The fusion proteins employ the α and β chains of a heterodimeric hormone or a portion thereof as a scaffold to which a polypeptide of the present invention is linked. An example of a heterodimeric proteinaceous hormone is the human chorionic gonadotropin (hCG) hormone, a stable secreted protein with a long half-life. Examples of hybrid proteins employing hCG can be found in U.S. Pat. No. 6,194,177, to Campbell et al., the entire contents of which are incorporated by reference herein.

In general, the hybrid proteins of the first and the sixth aspects of the invention include at least two polypeptide chains, where each polypeptide chain includes at least one polypeptide of the present invention linked to a subunit of a heterodimeric proteinaceous hormone, or a fragment thereof. Examples of proteinaceous heterodimeric hormones for use in this invention include but are not limited to FSH, inhibin, TSH, hCG, and LH.

In some embodiments, one of the subunits of the heterodimeric proteinaceous hormone in the hybrid protein comprises one or more alterations which reduce or eliminate the biological activity of the hormone, while preserving the ability of the altered subunit to dimerize with another subunit of the hormone. In some embodiments, an altered subunit is an alpha subunit of hCG which comprises a deletion of amino acids 88-92 (del 88-92), named alpha des88-92, thereby rendering the hCG biologically inactive; however, preserving the ability of the alpha subunit to dimerize with the beta subunit of hCG (removal of just five residues at the extreme carboxyl-terminus of a subunit of hCG can effectively eliminate its biological activity while preserving its capability to form heterodimers). In another embodiment, an altered subunit is an alpha subunit which comprises substitution of a cysteine residue at amino acid position 26 with an alanine (C26A). In another embodiment, an altered subunit is an alpha subunit comprising a deletion of amino acids 88-92 (del 88-92) and substitution of a cysteine residue at amino acid position 26 with an alanine (C26A). In another embodiment, an altered subunit is a beta subunit comprising a deletion of amino acids 104-145 (del 104-145). The hybrid proteins of the invention may comprise: a) an altered alpha subunit and an unaltered beta subunit; b) an altered alpha subunit and an altered beta subunit; c) an unaltered alpha subunit and an altered beta subunit; or d) an unaltered alpha subunit and an unaltered beta subunit.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP002 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP002 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table 1.

Specific, non-conservative mutations can be also introduced in the polypeptides of the invention with different purposes. Mutations reducing the affinity of INSP002 polypeptide may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Preferred alternative, synonymous groups for amino acids derivatives included in peptide mimetics are those defined in Table 2. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Typically, greater than 30% identity between two polypeptides is considered to be an indication of functional equivalence. Preferably, functionally equivalent polypeptides of the first aspect of the invention have a degree of sequence identity with the INSP002 polypeptide, or with active fragments thereof, of greater than 35%. More preferred polypeptides have degrees of identity of greater than 35%, 40%, 50%, 60%, 70%, 80%, 90%, 95%, 98%, 99% or more, respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium search database may be used (see co-pending United Kingdom patent application PCT/GB01/01105) to identify polypeptides of presently-unknown function which, while having low sequence identity as compared to the INSP002 polypeptides, are predicted to have secreted molecule activity, by virtue of sharing significant structural homology with the INSP002 polypeptide sequences. By “significant structural homology” is meant that the Inpharmatica Genome Threader predicts two proteins to share structural homology with a certainty of 10% and above.

The polypeptides of the first aspect of the invention also include fragments of the INSP002 polypeptides and fragments of the functional equivalents of the INSP002 polypeptides, provided that those fragments retain cystine knot fold cytokine activity, preferably the activity of a member of the DAN cystine knot fold subfamily or have an antigenic determinant in common with the INSP002 polypeptides.

As used herein, the term “fragment” refers to a polypeptide having an amino acid sequence that is the same as part, but not all, of the amino acid sequence of the INSP002 polypeptides or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Nucleic acids according to the invention are preferably 10-400 nucleotides in length, preferably 50-350 nucleotides, preferably 100-300, preferably 150-250, preferably 175-225 nucleotides in length. Polypeptides according to the invention are preferably 5-150 amino acids in length, preferably 10-130, preferably 25-120, preferably 50-100 amino acids in length.

Fragments of the full length INSP002 polypeptides may consist of combinations of 1 or 2 neighbouring exon sequences in the INSP002 polypeptide sequences, respectively. These exons may be combined with further mature fragments according to the invention. For example, such combinations include exons 1 and 2, exons 1 and 3 and so on. Such fragments are included in the present invention. Fragments may also consist of combinations of different domains of the INSP002 protein.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known secreted proteins.

Preferably, the affinity is at least 1.5-fold, 2-fold, 5-fold 10-fold, 100-fold, 10³-fold, 10⁴-fold, 10⁵-fold or 10⁶-fold greater for a polypeptide of the invention than for known secreted proteins such as cystine knot fold cytokines and in particular such as members of the DAN subfamily.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second aspect of the invention are those which encode the polypeptide sequences recited in SEQ ID NO:2, SEQ ID NO:4, SEQ ID NO:7, SEQ ID NO:6, SEQ ID NO:8, SEQ ID NO:14, SEQ ID NO:17, SEQ ID NO:19, SEQ ID NO:21, SEQ ID NO:23, SEQ ID NO:25, SEQ ID NO:27 and functionally equivalent polypeptides. Preferred nucleic acid molecules of the seventh and eighth aspects of the invention are those which encode the polypeptide sequence recited in SEQ ID NO:17, SEQ ID NO:19, SEQ ID NO:21, SEQ ID NO:23, SEQ ID NO:25, SEQ ID NO:27 and functionally equivalent polypeptides.

These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes the polypeptide of SEQ ID NO:2 may be identical to the coding sequence of the nucleic acid molecule shown in SEQ ID NO:1 (nucleotides 152 to 475), as recited in SEQ ID NO:12. A nucleic acid molecule which encodes the polypeptide of SEQ ID NO:7 may be identical to the coding sequence of the nucleic acid molecule shown in SEQ ID NO: 1 (nucleotides 218 to 475), as recited in SEQ ID NO:10. A nucleic acid molecule which encodes the polypeptide of SEQ ID NO:4 may be identical to the coding sequence of the nucleic acid molecule shown in SEQ ID NO:3. A nucleic acid molecule which encodes the polypeptide of SEQ ID NO: 6 may be identical to the coding sequence of the nucleic acid molecule shown in SEQ ID NO:5 (nucleotides 152 to 721), as recited in SEQ ID NO:11. A nucleic acid molecule which encodes the polypeptide of SEQ ID NO: 8 may be identical to the coding sequence of the nucleic acid molecule shown in SEQ ID NO:5 (nucleotides 218 to 721), as recited in SEQ ID NO:9. A nucleic acid molecule which encodes the polypeptide of SEQ ID NO:14 may be identical to the coding sequence of the nucleic acid molecule shown in SEQ ID NO:13 (nucleotides 69 to 719), as recited in SEQ ID NO:15. A nucleic acid molecule which encodes the polypeptide of SEQ ID NO:17 may be identical to the coding sequence of the nucleic acid molecule shown in SEQ ID NO:16. A nucleic acid molecule which encodes the polypeptide of SEQ ID NO:19 may be identical to the coding sequence of the nucleic acid molecule shown in SEQ ID NO:18. A nucleic acid molecule which encodes the polypeptide of SEQ ID NO:21 may be identical to the coding sequence of the nucleic acid molecule shown in SEQ ID NO:20. A nucleic acid molecule which encodes the polypeptide of SEQ ID NO:23 may be identical to the coding sequence of the nucleic acid molecule shown in SEQ ID NO:22. A nucleic acid molecule which encodes the polypeptide of SEQ ID NO:25 may be identical to the coding sequence of the nucleic acid molecule shown in SEQ ID NO:24. A nucleic acid molecule which encodes the polypeptide of SEQ ID NO:27 may be identical to the coding sequence of the nucleic acid molecule shown in SEQ ID NO:26.

These molecules also may have a different sequence which, as a result of the degeneracy of the genetic code, encodes a polypeptide of SEQ ID NO:2, SEQ ID NO:4, SEQ ID NO: 7, SEQ ID NO:6, SEQ ID NO: 8, SEQ ID NO:14, SEQ ID NO:17, SEQ ID NO:19, SEQ ID NO:21, SEQ ID NO:23, SEQ ID NO:25, SEQ ID NO:27. Such nucleic acid molecules that encode the polypeptide of SEQ ID NO:2, SEQ ID NO:4, SEQ ID NO:7, SEQ ID NO:6, SEQ ID NO:8, SEQ ID NO:14, SEQ ID NO:17, SEQ ID NO:19, SEQ ID NO:21, SEQ ID NO:23, SEQ ID NO:25, SEQ ID NO:27 may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or seventh aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP002 polypeptides (SEQ ID NO:2, SEQ ID NO:4, SEQ ID NO:7, SEQ ID NO:6, SEQ ID NO:8 and SEQ ID NO:14, SEQ ID NO:17, SEQ ID NO:19, SEQ ID NO:21, SEQ ID NO:23, SEQ ID NO:25, SEQ ID NO:27) and nucleic acid molecules that are substantially complementary to such nucleic acid molecules. Preferably, a nucleic acid molecule according to this aspect of the invention comprises a region that is at least 80% identical over its entire length to: the coding sequences for SEQ ID NO:2 and SEQ ID NO:7 given in SEQ ID NO:1, SEQ ID NO:10 and SEQ ID NO:12; the coding sequence for SEQ ID NO:4 given in SEQ ID NO:3; the coding sequences for SEQ ID NO:6 and SEQ ID NO:8 given in SEQ ID NO:5, SEQ ID NO:9 and SEQ ID NO:11; or the coding sequences for SEQ ID NO:14 given in SEQ ID NO:13 and SEQ ID NO:15; the coding sequence for SEQ ID NO:17 given in SEQ ID NO:16; the coding sequence for SEQ ID NO:19 given in SEQ ID NO:18; the coding sequence for SEQ ID NO:21 given in SEQ ID NO:20; the coding sequence for SEQ ID NO:23 given in SEQ ID NO:22; the coding sequence for SEQ ID NO:25 given in SEQ ID NO:24; the coding sequence for SEQ ID NO:27 given in SEQ ID NO:26; or is a nucleic acid molecule that is complementary thereto. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the INSP002 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP002 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP002 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:9, SEQ ID NO:10, SEQ ID NO:11, SEQ ID NO:12, SEQ ID NO:13 or SEQ ID NO:15, SEQ ID NO:16, SEQ ID NO:18, SEQ ID NO:20, SEQ ID NO:22, SEQ ID NO:24, SEQ ID NO:26), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al.,[supra]. Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSportl™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk− or aprt±cells, respectively.

Also, antimetabolite, antibiotic or herbicide resistance can be used as the basis for selection; for example, dihydrofolate reductase (DHFR) that confers resistance to methotrexate (Wigler, M.. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

As indicated above, the present invention also provides novel targets and methods for the screening of drug candidates or leads. These screening methods include binding assays and/or functional assays, and may be performed in vitro, in cell systems or in animals. As indicated above, the present invention also provides novel targets and methods for the screening of drug candidates or leads. These screening methods include binding assays and/or functional assays, and may be performed in vitro, in cell systems or in animals. In this regard, a particular object of this invention resides in the use of an INSP002 polypeptide as a target for screening candidate drugs for treating or preventing cystine-knot fole related disorders.

Another object of this invention resides in methods of selecting biologically active compounds, said methods comprising contacting a candidate compound with a INSP002 gene or polypeptide, and selecting compounds that bind said gene or polypeptide.

A further other object of this invention resides in methods of selecting biologically active compounds, said method comprising contacting a candidate compound with recombinant host cell expressing a INSP002 polypeptide with a candidate compound, and selecting compounds that bind said INSP002 polypeptide at the surface of said cells and/or that modulate the activity of the INSP002 polypeptide.

A “biologically active” compound denotes any compound having biological activity in a subject, preferably therapeutic activity, more preferably a compound having cystine-knot fold cytokine family activity, and further preferably a compound that can be used for treating INSP002 related disorders, or as a lead to develop drugs for treating a cystine-knot fold cytokine family disorder. A “biologically active” compound preferably is a compound that modulates the activity of INSP002.

The above methods may be conducted in vitro, using various devices and conditions, including with immobilized reagents, and may further comprise an additional step of assaying the activity of the selected compounds in a model of cystine-knot fold cytokine related disorder, such as an animal model.

Preferred selected compounds are agonists of INSP002, i.e., compounds that can bind to INSP002 and mimic the activity of an endogenous ligand thereof.

A further object of this invention resides in a method of selecting biologically active compounds, said method comprising contacting in vitro a test compound with a INSP002 polypeptide according to the present invention and determining the ability of said test compound to modulate the activity of said INSP002 polypeptide.

A further object of this invention resides in a method of selecting biologically active compounds, said method comprising contacting in vitro a test compound with a INSP002 gene according to the present invention and determining the ability of said test compound to modulate the expression of said INSP002 gene, preferably to stimulate expression thereof.

In another embodiment, this invention relates to a method of screening, selecting or identifying active compounds, particularly compounds active on multiple sclerosis or related disorders, the method comprising contacting a test compound with a recombinant host cell comprising a reporter construct, said reporter construct comprising a reporter gene under the control of a INSP002 gene promoter, and selecting the test compounds that modulate (e.g. stimulate or reduce, preferably stimulate) expression of the reporter gene.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Binding to a target gene or polypeptide provides an indication as to the ability of the compound to modulate the activity of said target, and thus to affect a pathway leading to cystine knot fold cytokine family related disorder in a subject. The determination of binding may be performed by various techniques, such as by labelling of the candidate compound, by competition with a labelled reference ligand, etc. For in vitro binding assays, the polypeptides may be used in essentially pure form, in suspension, immobilized on a support, or expressed in a membrane (intact cell, membrane preparation, liposome, etc.).

The cells used in the assays may be any recombinant cell (i.e., any cell comprising a recombinant nucleic acid encoding a INSP002 polypeptide) or any cell that expresses an endogenous INSP002 polypeptide. Examples of such cells include, without limitation, prokaryotic cells (such as bacteria) and eukaryotic cells (such as yeast cells, mammalian cells, insect cells, plant cells, etc.). Specific examples include E. coli, Pichia pastoris, Hansenula polymorpha, Schizosaccharomyces pombe, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:
(a) contacting a cell expressing (optionally on on the surface thereof) the polypeptide according to the first aspect of the invention, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

Methods for generating detectable signals in the types of assays described herein will be known to those of skill in the art. A particular example is cotransfecting a construct expressing a polypeptide according to the invention, or a fragment such as the LBD, in fusion with the GAL4 DNA binding domain, into a cell together with a reporter plasmid, an example of which is pFR-Luc (Stratagene Europe, Amsterdam, The Netherlands). This particular plasmid contains a synthetic promoter with five tandem repeats of GAL4 binding sites that control the expression of the luciferase gene. When a potential ligand is added to the cells, it will bind the GAL4-polypeptide fusion and induce transcription of the luciferase gene. The level of the luciferase expression can be monitored by its activity using a luminescence reader (see, for example, Lehman et al. JBC 270, 12953, 1995; Pawar et al. JBC, 277, 39243, 2002).

A further preferred method for identifying an agonist or antagonist of a polypeptide of the invention comprises:
(a) contacting a labelled or unlabeled compound with the polypeptide immobilized on any solid support (for example beads, plates, matrix support, chip) and detection of the compound by measuring the label or the presence of the compound itself; or
(b) contacting a cell expressing on the surface thereof the polypeptide, by means of artificially anchoring it to the cell membrane, or by constructing a chimeric receptor being associated with a second component capable of providing a detectable signal in response to the binding of a compound to the polypeptide, with a compound to be screened under conditions to permit binding to the polypeptide; and
(c) determining whether the compound binds to and activates or inhibits the polypeptide by comparing the level of a signal generated from the interaction of the compound with the polypeptide with the level of a signal in the absence of the compound.

For example, a method such as FRET detection of ligand bound to the polypeptide in the presence of peptide co-activators (Norris et al, Science 285, 744, 1999) might be used.

A further preferred method for identifying an agonist or antagonist of a polypeptide of the invention comprises:
(a) contacting a cell expressing (optionally on the surface thereof) the polypeptide, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:

determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:
(a) incubating a labelled ligand with a whole cell expressing a polypeptide according to the invention on the cell surface, or a cell membrane containing a polypeptide of the invention,
(b) measuring the amount of labelled ligand bound to the whole cell or the cell membrane;
(c) adding a candidate compound to a mixture of labelled ligand and the whole cell or the cell membrane of step (a) and allowing the mixture to attain equilibrium;
(d) measuring the amount of labelled ligand bound to the whole cell or the cell membrane after step (c); and
(e) comparing the difference in the labelled ligand bound in step (b) and (d), such that the compound which causes the reduction in binding in step (d) is considered to be an agonist or antagonist.

Similarly, there is provided a method of screening for a polypeptide antagonist or agonist compound which comprises the steps of:

(a) incubating a labelled ligand with a polypeptide according to the invention on any solid support or the cell surface, or a cell membrane containing a polypeptide of the invention.

(b) measuring the amount of labelled ligand bound to the polypeptide on the solid support, whole cell or the cell membrane;

(c) adding a candidate compound to a mixture of labelled ligand and immobilized polypeptide on the solid support, the whole cell or the cell membrane of step (a) and allowing the mixture to attain equilibrium;

(d) measuring the amount of labelled ligand bound to the immobilized polypeptide or the whole cell or the cell membrane after step (c); and

(e) comparing the difference in the labelled ligand bound in step (b) and (d), such that the compound which causes the reduction in binding in step (d) is considered to be an agonist or antagonist.

The polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner.

Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signaling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance (e.g. supplied by Biacore AB, Uppsala Sweden) and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

In another embodiment, this invention relates to the use of a INSP002 polypeptide or fragment thereof, whereby the fragment is preferably a INSP002 gene-specific fragment, for isolating or generating an agonist or stimulator of the INSP002 polypeptide for the treatment of an cystine knot fold cytokine associated disorder, wherein said agonist or stimulator is selected from the group consisting of:

1. a specific antibody or fragment thereof including: a)a chimeric, b) a humanized or c) a fully human antibody, as well as;

2. a bispecific or multispecific antibody,

3. a single chain (e.g. scFv) or

4. single domain antibody, or

5. a peptide- or non-peptide mimetic derived from said antibodies or

6. an antibody-mimetic such as a) an anticalin or b) a fibronectin-based binding molecule (e.g. trinectin or adnectin).

The generation of peptide- or non-peptide mimetics from antibodies is known in the art (Saragovi et al., 1991 and Saragovi et al., 1992).

Anticalins are also known in the art (Vogt et al., 2004). Fibronectin-based binding molecules are described in U.S. Pat. No. 6,818,418 and WO2004029224.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

As mentioned above, it is envisaged that the various moieties of the invention (i.e. the polypeptides, a nucleic acid molecules, vectors, a host cells, ligands, compounds) may be useful in the therapy or diagnosis of diseases. To assess the utility of the moieties of the invention for treating or diagnosing a disease one or more of the following assays may be carried out. Note that although some of the following assays refer to the test compound as being a protein/polypeptide, a person skilled in the art will readily be able to adapt the following assays so that the other moieties of the invention may also be used as the “test compound”.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intraarterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′-O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent. Where the aforementioned polypeptide or nucleic acid molecule is one that is up-regulated, vaccine development can involve the raising of antibodies or T cells against such agents (as described in WO00/29428).

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;

wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application W095/251116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly cell proliferative disorders, autoimmune/inflammatory disorders, cardiovascular disorders, neurological disorders, developmental disorders, metabolic disorders, infections and other pathological conditions. The disease or disorder is preferably a disease in which aberrant levels of a cystine knot fold cytokine, preferably of a member of the DAN subfamily, are implicated. The disease or disorder may also be one in which aberrant levels of a ligand of a cystine knot fold cytokine, preferably a ligand of a member of the DAN subfamily, are implicated. For example, the disease or disorder may be one in which aberrant levels of a TGFBeta superfamily member are implicated. In particular, the disease or disorder may be one in which BMPs are implicated, such as neuropathies, nephropathies such as diabetic mephropathy, cancer, wound healing, fibrosis, osteopenia, osteoporosis, fractures and sclerosteosis. Various aspects and embodiments of the present invention will now be described in more detail by way of example, with particular reference to INSP002 polypeptides.

It will be appreciated that modification of detail may be made without departing from the scope of the invention.

### BRIEF DESCRIPTION OF THE FIGURES

FIG. 1: Results from BLAST against NCBI non-redundant database using combined SEQ ID NO:2 and SEQ ID NO:4 polypeptide sequence.

FIG. 2 Alignment generated by BLAST between combined SEQ ID NO:2 and SEQ ID NO:4 polypeptide sequence and the closet related sequence, Homo sapiens cerberus-related 1 protein.

FIG. 3: Top four NCBI-nr and NCBI-nt database BLAST hits against INSP002 on 26th November 2002

FIG. 4: Alignment of INSP002 with AK095926.1

FIG. 5: Alignment of INSP002 with IMAGE: 4558384

FIG. 6: INSP002 nucleotide sequence with translation

FIG. 7: INSP002 partial cloned sequence with translation

FIG. 8: Map of PCRII-TOPO-INSP002 partial

FIG. 9: Alignment of INSP002 prediction (top) and partial cloned sequence (bottom)

FIG. 10: Nucleotide sequence and translation of cDNA insert in Image:4558384

FIG. 11: Alignment of sequences of INSP002 prediction (top) with IMAGE: 4558384 (BC025333.1) (bottom)

FIG. 12: Nucleotide sequence and translation of INSP002V generated by PCR from Image 4558384

FIG. 13: Map of pCR4blunt-TOPO-INSP002V

FIG. 14: Comparison between INSP002 Prediction (top) and INSP002V Variant sequence (bottom)

FIG. 15: Map of expression vector pEAK12d

FIG. 16: Map of Gateway vector pDONR201

FIG. 17: Map of pEAK12d-INSP002-V-6HIS

FIG. 18: Sequence of full-length INSP002 cloned from heart.

FIG. 19: Map of plasmid pCR4-blunt TOPO-INSP002FL encoding full-length INSP002 cloned from heart.

FIG. 20: IL-11 assay using ELISA kit as described in the protocol. FIG. 20A: 12 500; 25 000 and 50 000 cells/well in the presence and absence of TGFb. The optimal cell amount is 50 000 cells/well. FIG. 20B: IL-11 assay in the presence of different concentrations of TGFb-dose response curve. IC50 is about 0.5 ng/ml. 5 ng/ml dose was chosen for further experiments because of the robustness of the assay at this concentration. FIG. 20C: IL-11 ELISA in the presence of positive (TGFb monoclonal antibodies from R&D systems) and negative (OPG-Fc, Serono) controls.

FIG. 21: IL-11 assay using ELISA kit. INSP002-Fc was expressed in HEK 293 cells as described in the protocol and partially purified. Not purified conditioned media, partially purified protein and conditioned media from cell without transfection were used in the assay in different dilutions (⅕; 1/10; 1/20; 1/100).

FIG. 22: An assay was performed as described in FIG. 21, but the protein was purified using MonoQ column (conventional chromatography).

FIG. 23: An assay was performed as described in FIG. 21, but protein was purified using protein A column.

FIG. 24: Right panel: INSP002-Fc was purified using protein A column. Left panel: INSP002-6His was expressed in E. Coli and refolded from inclusion bodies.

FIG. 25: IL-11 assay in the presence of different concentrations of INSP002-Fc. High doses ( 1/10 and 1/50) inhibited IL-11 production because they inhibited cell proliferation, as indicated in the corresponding cell proliferation assay (lower panel). 1/1000 dilution activated IL-11 production.

TABLE 1

| | | More Preferred |
|---|---|---|
| Amino Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE 2

| Amino Acid | Synonymous Groups |
|---|---|
| Ser | D-Ser, Thr, D-Thr, allo-Thr, Met, D-Met, Met(O), D-Met(O), L-Cys, |
| | D-Cys |
| Arg | D-Arg, Lys, D-Lys, homo-Arg, D-homo-Arg, Met, Ile, D-.Met, D-Ile, |
| | Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or L-1-oxazolidine-4- |
| | carboxylic acid |
| Thr | D-Thr, Ser, D-Ser, allo-Thr, Met, D-Met, Met(O), D-Met(O), Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, Trans-3,4, or 5- |
| | phenylproline, AdaA, AdaG, cis-3,4, or 5-phenylproline, Bpa, D-Bpa |
| Tyr | D-Tyr, Phe, D-Phe, L-Dopa, His, D-His |
| Cys | D-Cys, S--Me--Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, Ile, D-Ile, |
| | Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S--Me--Cys, Ile, D-Ile, Leu, D-Leu, Val, D-Val |

### EXAMPLES

### Example 1

### Comparison of INSP002 Protein with Proteins in Sequence Database

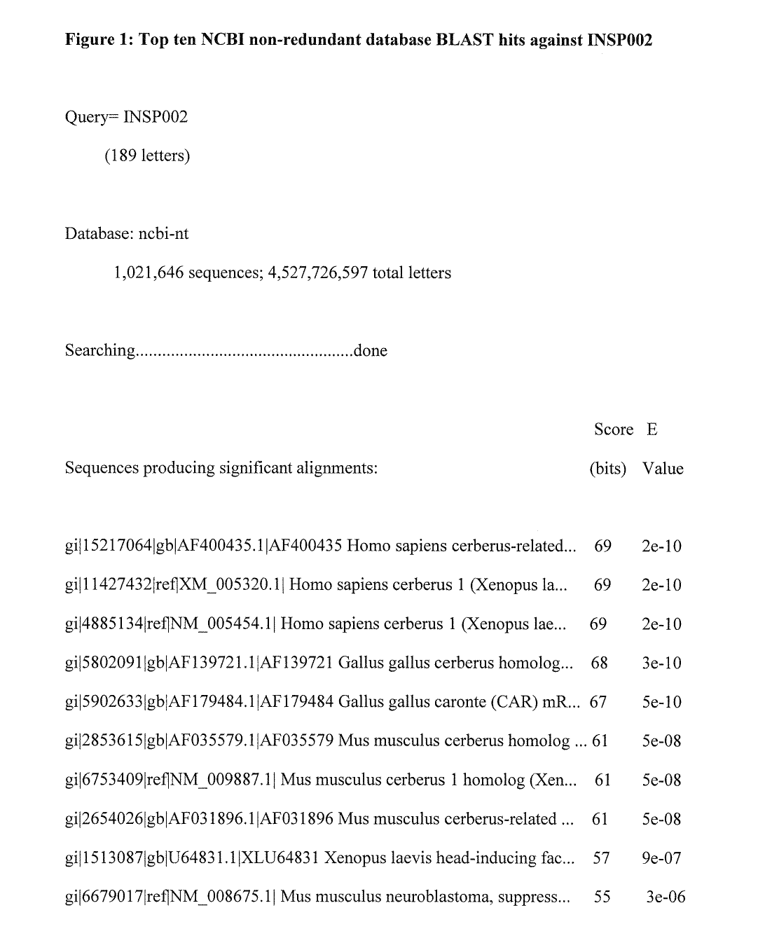

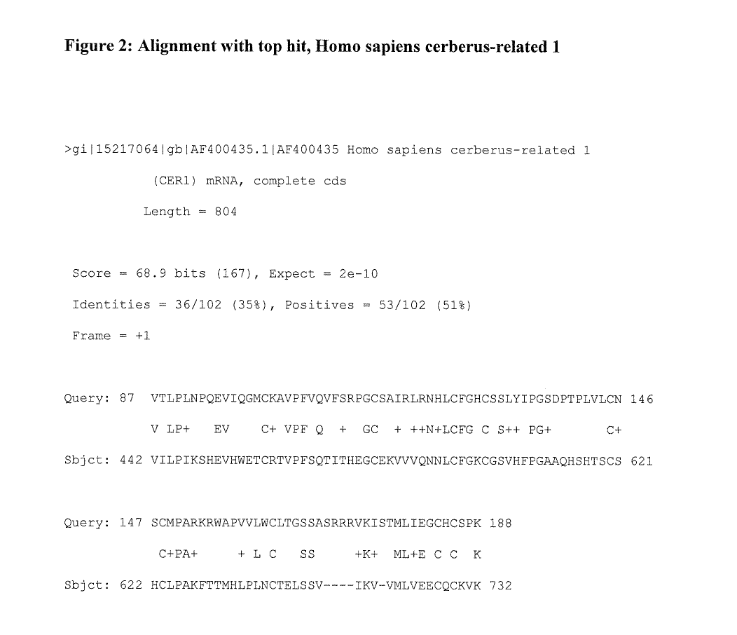

The polypeptide sequence derived from combining SEQ ID NO:2 and SEQ ID NO:4, which represents the translation of consecutive exons from INSP002 was used as a BLAST query against the NCBI non-redundant Sequence database. The top ten matches include sequences annotated as cerberus or cerberus related proteins, which are members of the cystine knot family, all of which align to the query sequence with highly significant E-values (2E⁻¹⁰ to 3E⁻⁰⁶) (FIG. 1). FIG. 2 shows the alignment of the INSP002 query sequence to the sequence of Homo sapiens cerberus-related 1 protein (Feng et al. 2001).

The polypeptide sequence derived from combining SEQ ID NO:2 and SEQ ID NO:4, which represents the translation of consecutive exons from INSP002 was inputted into SignalP V2.0.b2 (Nielsen et al. 1997 Protein Eng 1:1-6). The program predicted that the polypeptide sequence had a signal peptide. The most likely cleavage site for the signal peptide is to be found between residues 22 and 23 of the polypeptide sequence, INSP002, derived from combining SEQ ID NO:2 and SEQ ID NO:4.

The nucleotide sequence SEQ ID NO:1, encoding the polypeptide SEQ ID NO:2 exon 1, comprises of 5′ untranslated region (5′UTR) and protein coding sequence (CDS). The CDS starts at nucleotide 152.

### Example 2

### Repetition of BLAST Searches

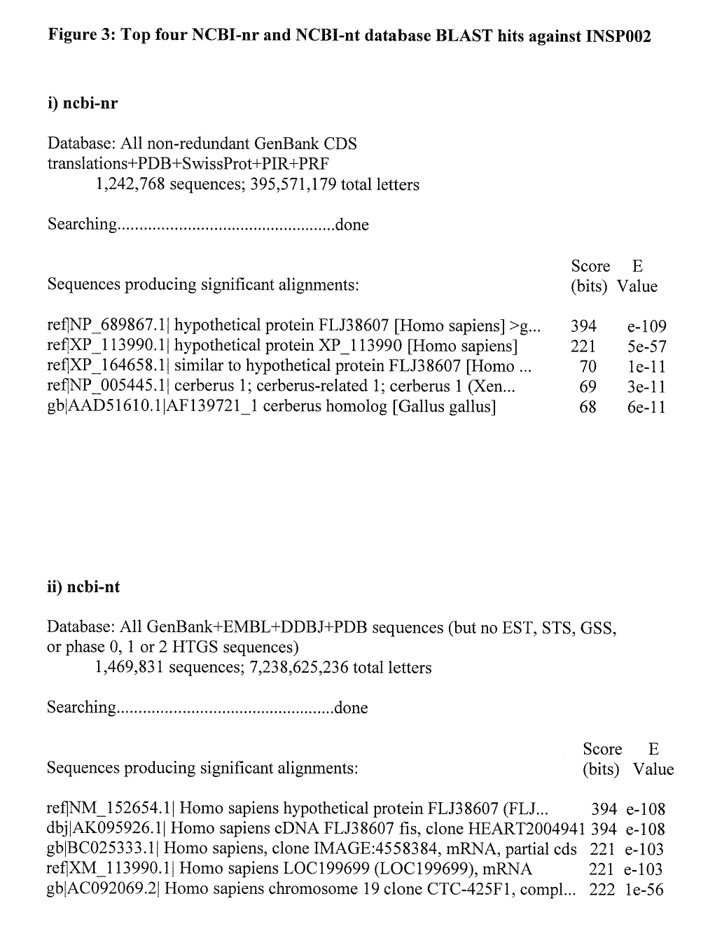

BLAST searches of the NCBI-nr and NCBI-nt databases were conducted on 26th Nov. 2002 using the polypeptide sequence of SEQ ID NO:6, derived from combining SEQ ID NO:2 and SEQ ID NO:4. The top four hits identified by these searches are shown in FIG. 3.

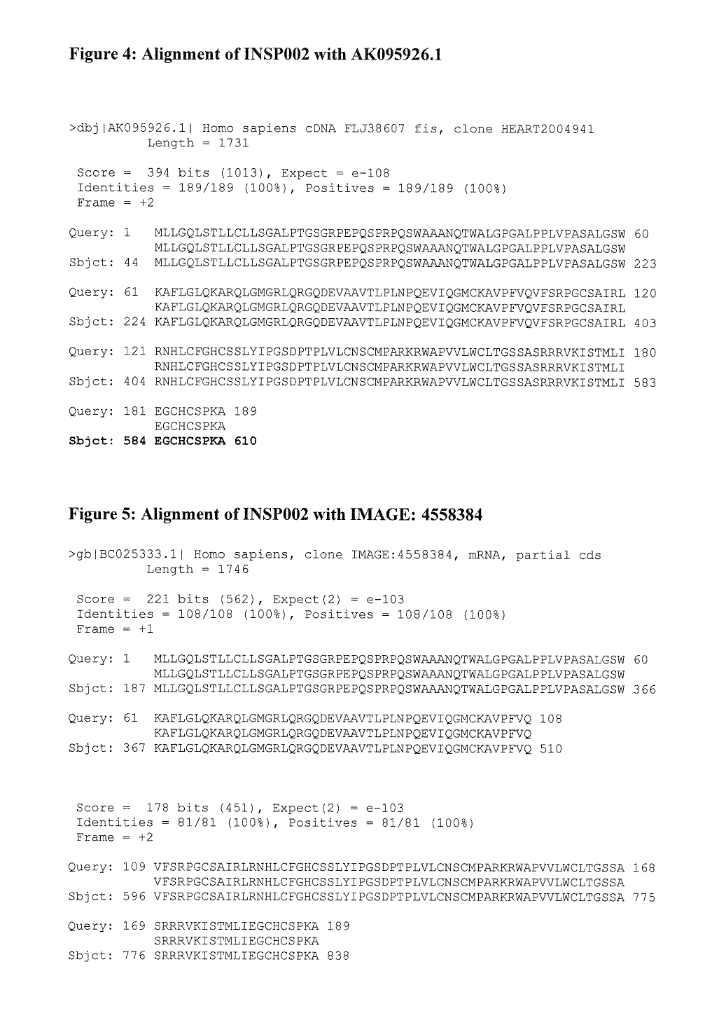

The searches revealed that the INSP002 polypeptide is identical to hypothetical protein FLJ38607 at the amino acid level and the corresponding nucleotide sequence AK095926, cloned from the heart and deposited on 16th Jul. 2002. FIG. 4 shows the alignment of the INSP002 query sequence with the protein derived from the AK095926 cDNA clone. The exon 1 and exon 2 splice junction predicted for INSP002 is proven experimentally by the existence of AK095926.

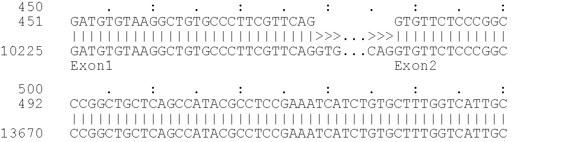

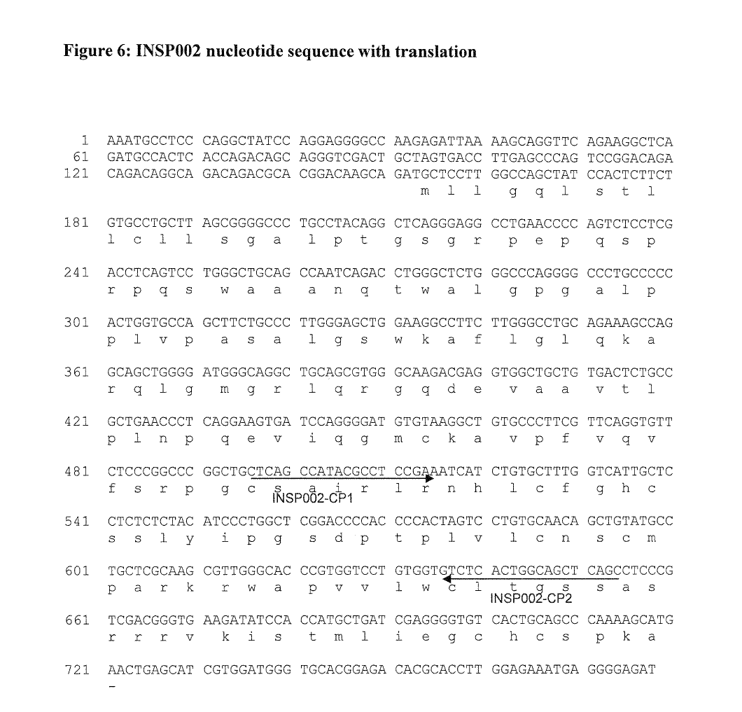

The searches also revealed that parts of the INSP002 are identical to IMAGE clone 4558384 (BC025333.1) deposited on 8th Mar. 2002. FIG. 5 shows the alignment of parts of the INSP002 query sequence with IMAGE clone 4558384.

### Example 3

### Partial Cloning of cDNA for INSP002

i) cDNA Libraries

Human cDNA libraries (in bacteriophage lambda (λ) vectors) were purchased from Stratagene or Clontech or prepared at the Serono Pharmaceutical Research Institute in λ ZAP or λ GT10 vectors according to the manufacturer's protocol (Stratagene). Bacteriophage λ DNA was prepared from small scale cultures of infected E. coli host strain using the Wizard Lambda Preps DNA purification system according to the manufacturer's instructions (Promega, Corporation, Madison Wis.) The list of libraries and host strains used is shown in Table I.

ii) PCR of Virtual cDNAs from Phage Library DNA

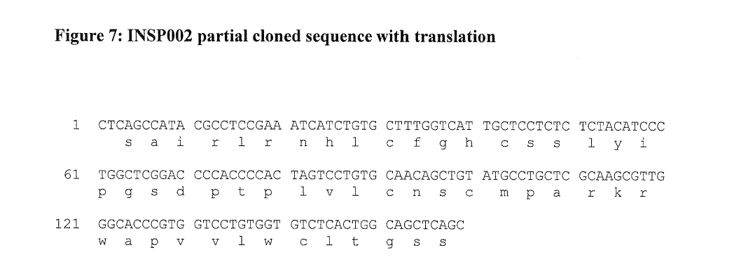

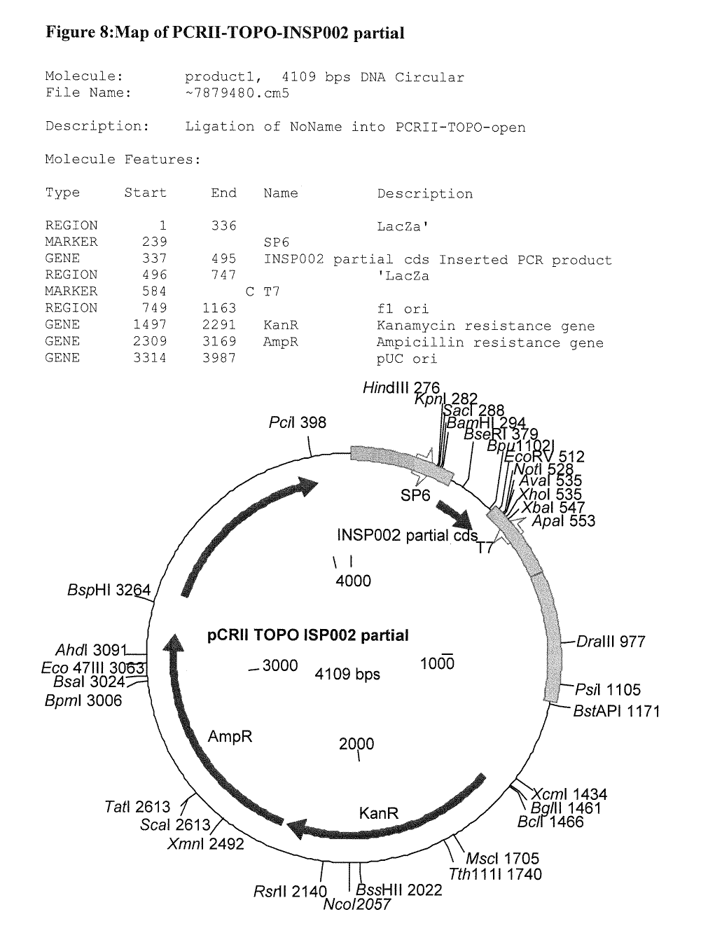

A partial cDNA encoding INSP002 (FIG. 6) was obtained as a PCR amplification product of 159 bp (FIG. 7) using gene specific cloning primers (INSP002-CP1 and INSP002-CP2, FIG. 6 and Table II). The PCR was performed in a final volume of 50 μl containing 1×AmpliTaq™ buffer, 200 μM dNTPs, 50 pmoles each of cloning primers, 2.5 units of AmpliTaq™ (Perkin Elmer) and 100 ng of each phage library DNA using an MJ Research DNA Engine, programmed as follows: 94° C., 1 min; 40 cycles of 94° C., 1 min, x ° C., and y min and 72° C., (where x is the lowest Tm−5° C. and y=1 min per kb of product); followed by 1 cycle at 72° C. for 7 min and a holding cycle at 4° C.

The amplification products were visualized on 0.8% agarose gels in 1×TAE buffer Invitrogen) and PCR products migrating at the predicted molecular mass were purified from the gel using the Wizard PCR Preps DNA Purification System (Promega). PCR products eluted in 50 μl of sterile water were either subcloned directly or stored at −20° C.

iii) Gene Specific Cloning Primers for PCR

Pairs of PCR primers having a length of between 18 and 25 bases were designed for amplifying the full length sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence INSP002 (little or no specific priming to other templates).

iv) Subcloning of PCR Products

PCR products were subcloned into the topoisomerase I modified cloning vector (pCR II TOPO) using the TOPO TA cloning kit purchased from the Invitrogen Corporation (cat. No. K4600-01 and K4575-01 respectively) using the conditions specified by the manufacturer. Briefly, 4 μl of gel purified PCR product from the human fetal kidney library (library number 12) amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing ampicillin (100 μg/ml) and incubated overnight at 37° C. Ampicillin resistant colonies containing cDNA inserts were identified by colony PCR.

v) Colony PCR

Colonies were inoculated into 50 μl sterile water using a sterile toothpick. A 10 μl aliquot of the inoculum was then subjected to PCR in a total reaction volume of 20 μl as described above, except the primers pairs used were SP6 and T7. The cycling conditions were as follows: 94° C., 2 min; 30 cycles of 94° C., 30 sec, 47° C., 30 sec and 72° C. for 1 min); 1 cycle, 72° C., 7 min. Samples were then maintained at 4° C. (holding cycle) before further analysis.

PCR reaction products were analyzed on 1% agarose gels in 1×TAE buffer. Colonies which gave the expected PCR product size (159 bp cDNA+187 bp due to the multiple cloning site or MCS) were grown up overnight at 37° C. in 5 ml L-Broth (LB) containing ampicillin (100 μg/ml), with shaking at 220 rpm at 37° C.

vi) Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with T7 primer and SP6 primer using the BigDye Terminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analysed on an Applied Biosystems 3700 sequencer.

vii) Identification of cDNA Libraries Containing INSP002

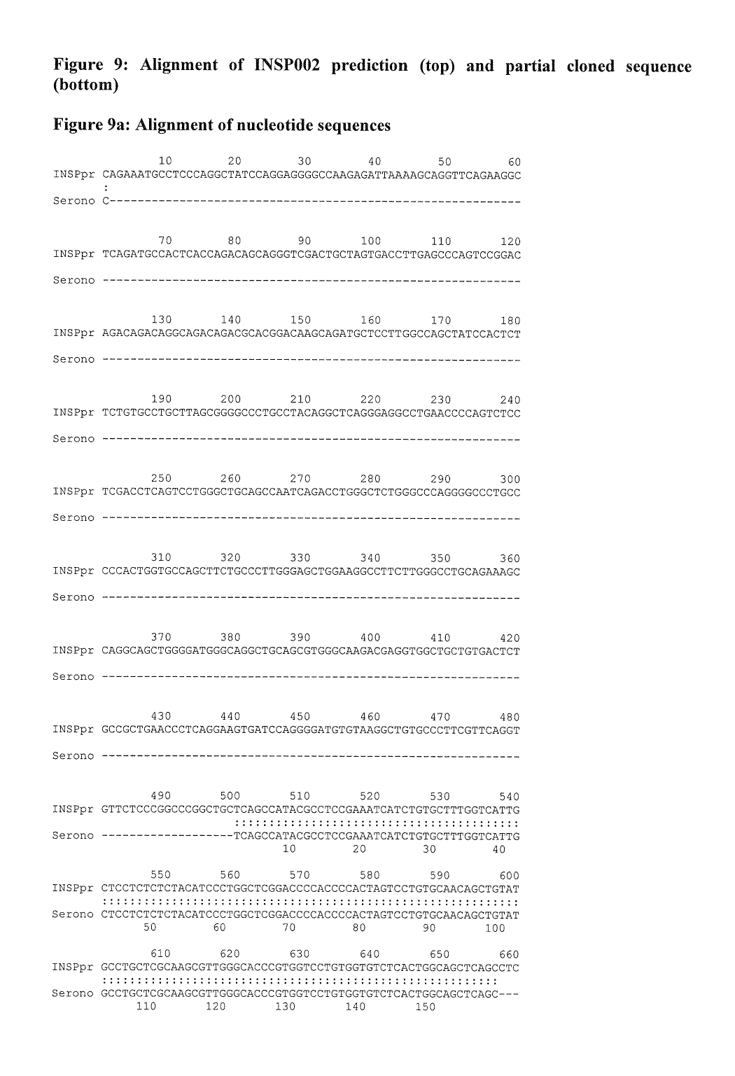

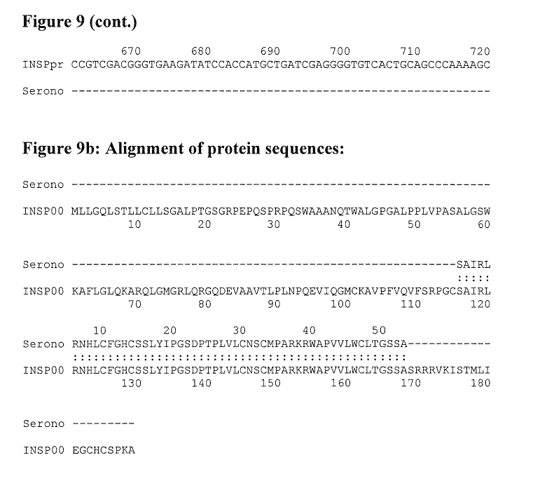

PCR products obtained with INSP002-CP1 and INSP002-CP2 and migrating at the correct size (159 bp) were identified in the cortex, colon, fetal lung and fetal kidney cDNA libraries (libraries 8, 9, 11 and 12). The sequence of the PCR product cloned in pCRII-TOPO vector is shown in FIG. 7, and the plasmid map (plasmid ID 13422) is in FIG. 8. The partial cDNA cloned is a portion of INSP002 exon 2, as shown by the alignment of the predicted INSP002 nucleotide sequence and the cloned partial nucleotide sequence in FIG. 9a and the alignment of the predicted INSP002 protein sequence and the cloned partial protein sequence in FIG. 9b.

### Example 4

### Generation of INSP002 ORF from Image: 4558384

Image clone 4558384 (in plasmid pOTB7) from retinoblastoma was purchased from Resgen (Invitrogen Corp). The E. coli stab of 4558384 was spread on an LB plate containing ampicillin (100 μg/ml) and grown up overnight at 37° C. Single ampicillin resistant colonies were inoculated into 5 ml LB containing ampicillin (100 μg/ml), and incubated with shaking at 220 rpm overnight at 37° C. Mini prep plasmid DNA was prepared and sequenced using SP6, T7, M13F, INSP002-CP1 and INSP002-CP2 primers as described in Example 3, vi).

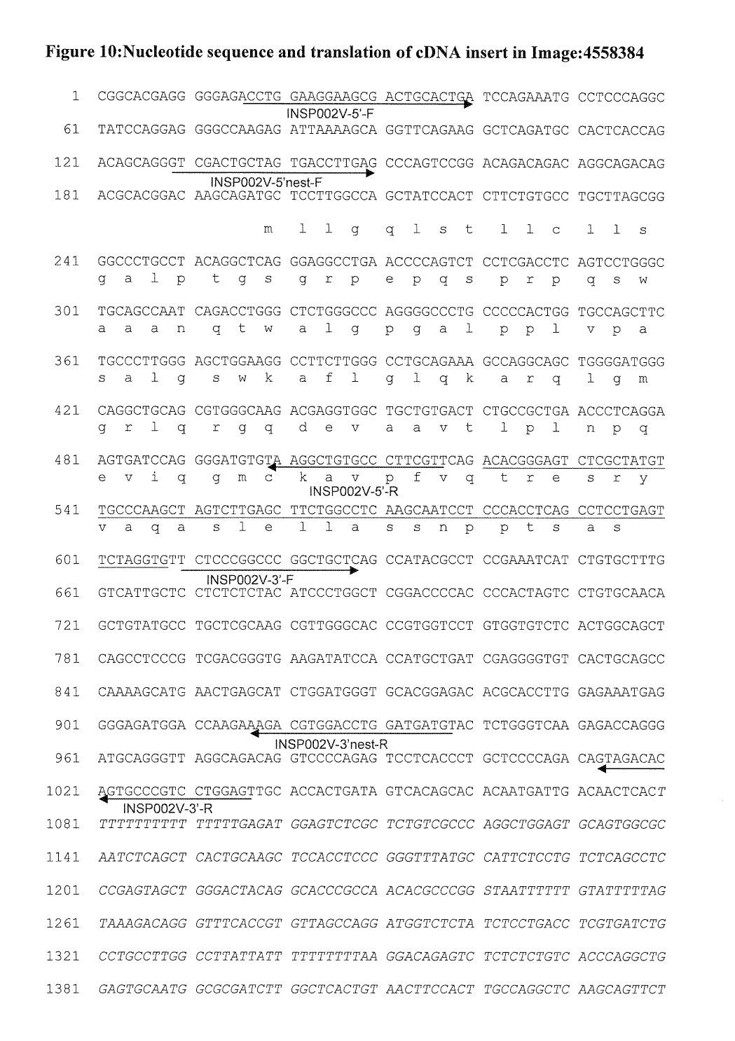

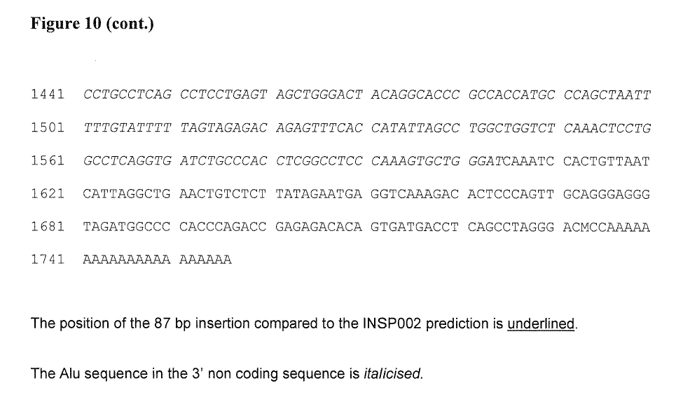

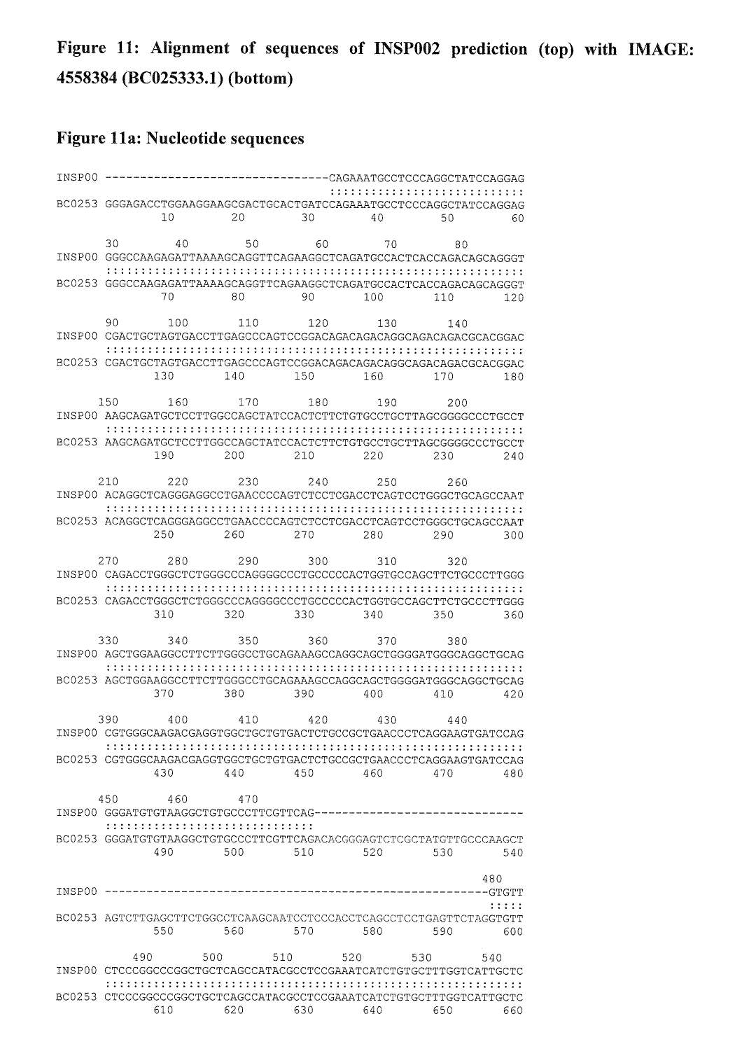

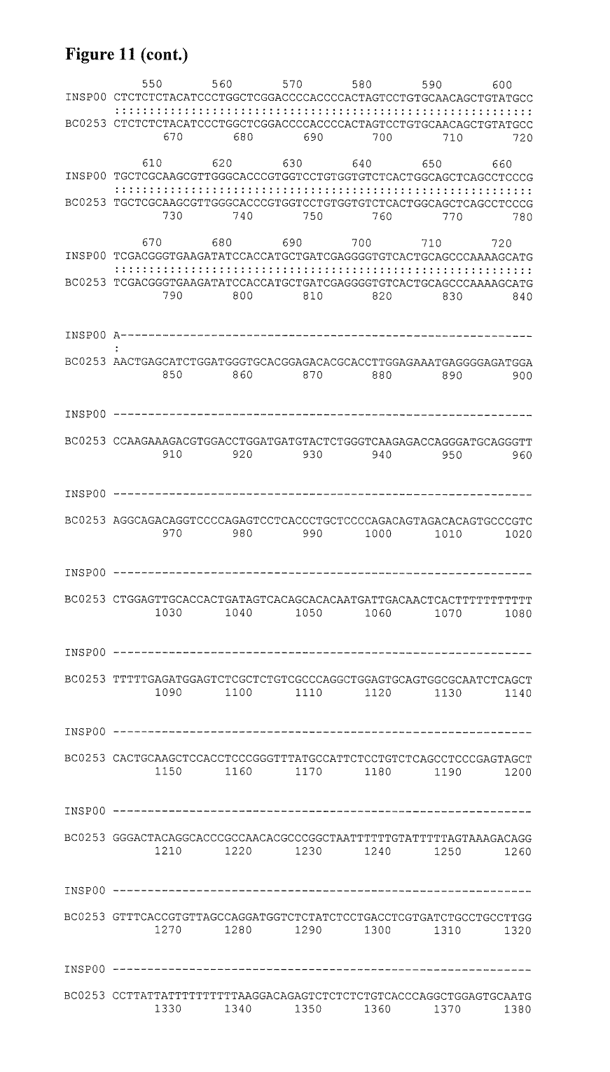

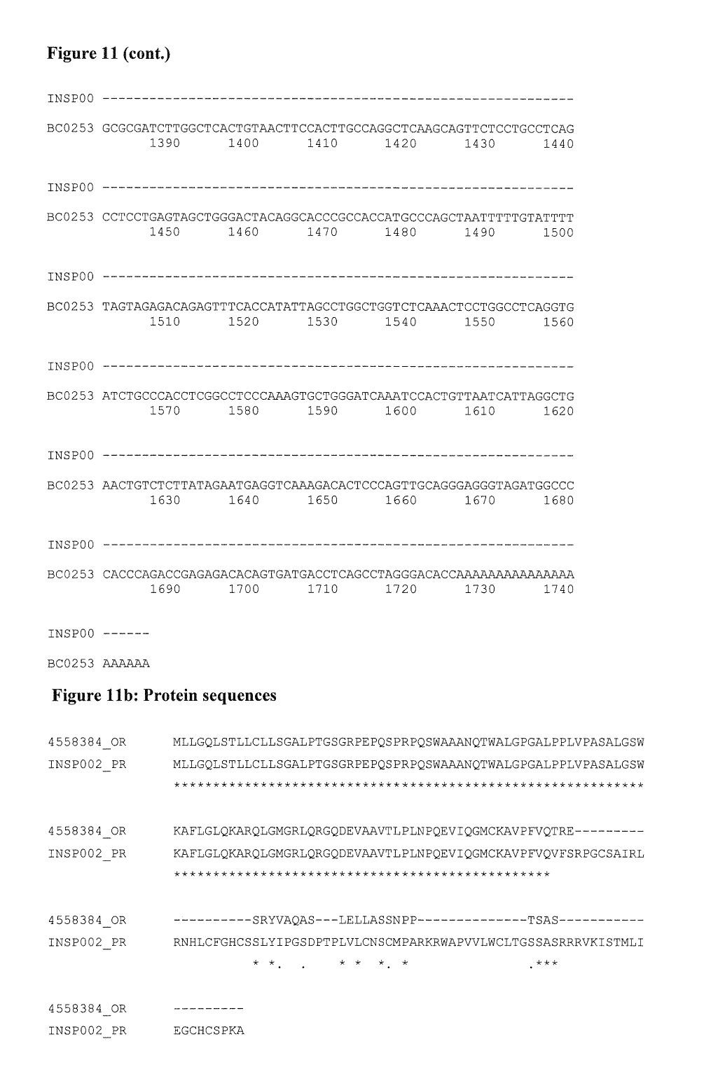

The sequence of the insert is shown in FIG. 10. The alignment of the-nucleotide and putative amino acid sequence of Image 4558384 cDNA with INSP002 is shown in FIG. 11. Image 4558384 cDNA appears to be a splice variant of INSP002. It contains an 87 bp insertion which introduces a frameshift and premature stop codon compared to the INSP002 predicted sequence. In addition the 3′ untranslated sequence also contains an Alu repeat indicative of genomic DNA contamination of the cDNA. Exons 2 and 4 of Image 4558384 are equivalent to INSP002 prediction exons 1 and 2. However, Image 4558384 incorporates an extra exon between exons 1 and 2 of the INSP002 prediction. The extra exon encodes a premature stop codon which prevents translation of the cystine knot domain. The splice boundaries in Image 4558384 are as follows:

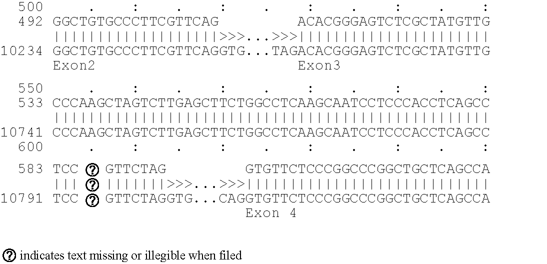

A PCR strategy was devised to remove the genomic DNA in order to generate a full length cDNA encoding the INSP002 ORF. PCR primers were designed to amplify the 5′ end (upstream) and 3′ end (downstream) of the INSP002 sequence which flanked the 87 bp insertion in the Image clone. The reverse primer for the upstream sequence and the forward primer for the downstream sequence contained complementary sequences at their 3′ and 5′ ends respectively to provide overlapping ends, so that the PCR products from each reaction could be mixed and annealed together, allowing amplification of the full length cDNA in a third PCR reaction, using a nested upstream forward primer and a nested reverse downstream primer.

The first PCR reaction to amplify the 5′ end of INSP002 (upstream of the 87 bp insertion) contained, in a final volume of 50 μl: 5 μl 10×Platinum Pfx buffer, 1.5 μl dNTPs (10 mM), 1 μl MgSO4 (50 mM), 1.5 μl of INSP002V-5′-F (10 μM), 1.5 μl INSP002V-5′-R (10 μM), 0.75 μl Platinum Pfx and 135 ng IMAGE:4558384 plasmid cDNA. The amplification conditions were 1 cycle of 94° C. for 2 min, 30 cycles of 94° C., 15 s and 68° C., 1 min; and 1 cycle of 68° C. for 7 min. The second PCR reaction to amplify the 3′ end of INSP002 (downstream of the 87 bp insertion) was performed under the same conditions except that the primers were: INSP002V-3′-F and INSP002V-3′-R.

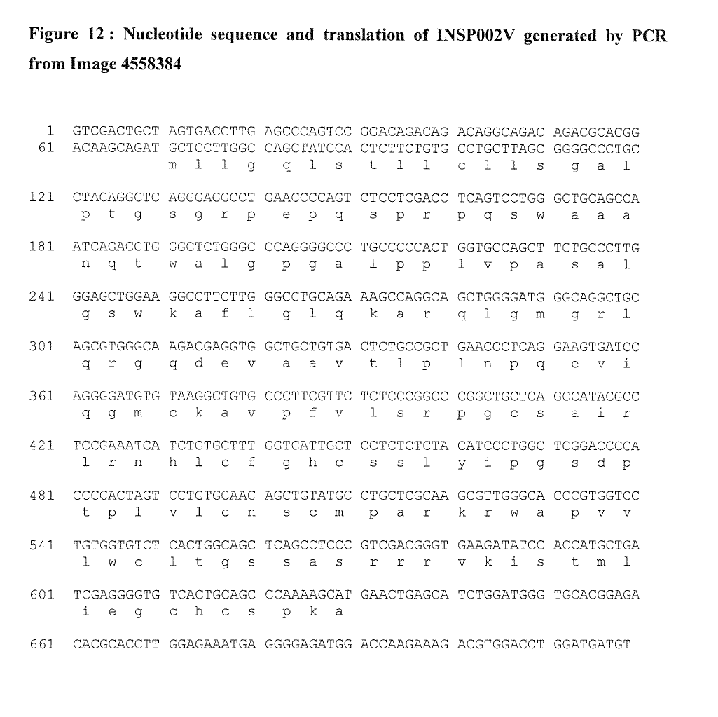

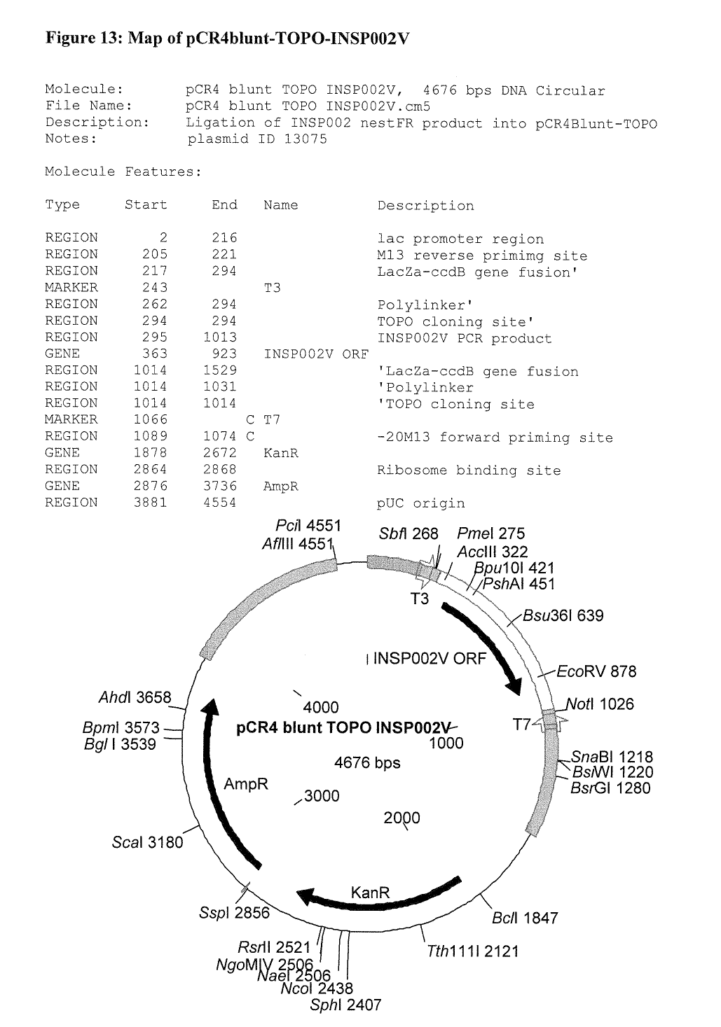

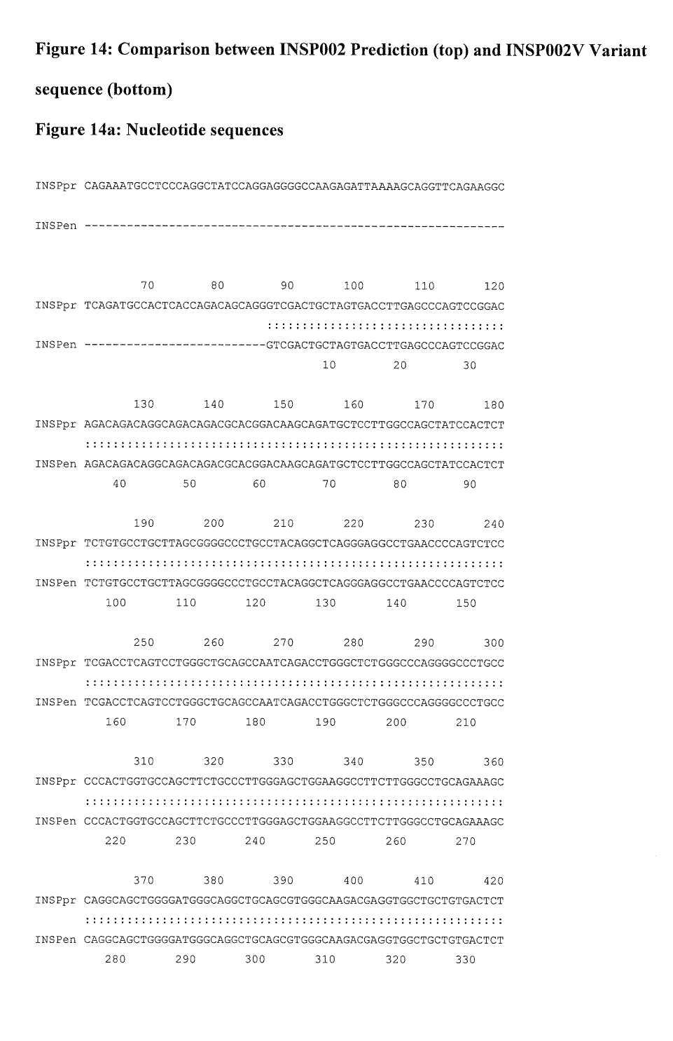

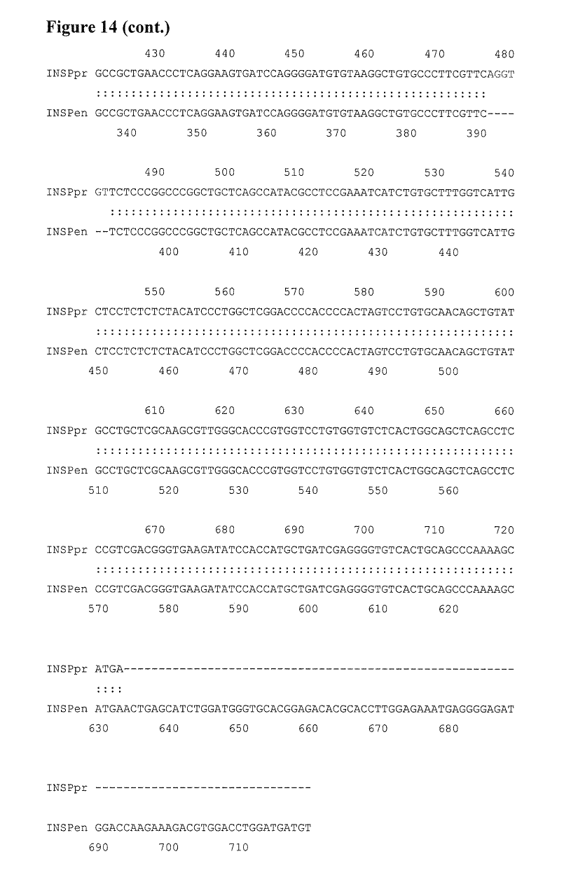

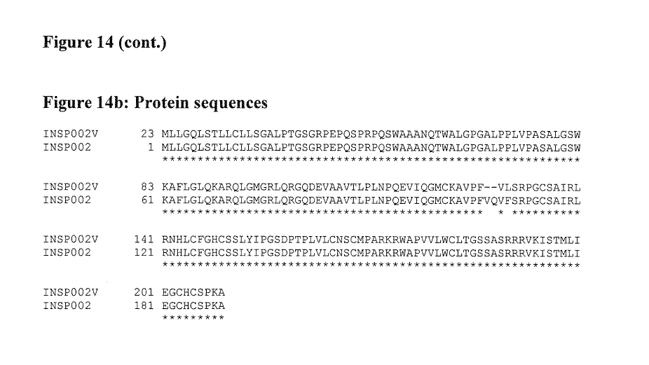

The amplification products were visualized on 0.8% agarose gels in 1×TAE buffer (Invitrogen). PCR products migrating at the predicted molecular mass (520 bp and 448 bp, for PCR 1 and 2 respectively) were purified from the gel using the Wizard PCR Preps DNA Purification System (Promega). PCR products were eluted in 50 μl of sterile water and the DNA concentration was measured using an Eppendorf BO photometer. Fifty ng of each purified PCR product was then used as a template for a nested PCR in a 50 μl reaction containing 5 μl 10×Platinum Pfx buffer, 1.5 μl dNTPs (10 mM), 1 μl MgSO4 (50 mM), 1.5 μl of INSP002V-5′nest-F (10 μM) and 1.5 μl INSP002V-3′nest-R (10 μM). The reaction mix was heated at 95° C. for 3 min and 0.75 μl Platinum Pfx polymerase added. The amplification conditions were as follows: 1 cycle of 94° C. for 2 min; 30 cycles of 94° C., 15 s; 61° C., 30 s and 68° C, 1 min; and 1 cycle of 68° C. for 7 min. PCR products migrating at the predicted molecular mass of 719 bp were purified from the gel using the Wizard PCR Preps DNA Purification System and eluted in 50 μl sterile water. Four μl of the purified PCR product was then ligated into pCR4 blunt TOPO vector as described in section 1.4. Ampicillin resistant colonies were tested for inserts by colony PCR using T3 and T7 primers as described in section 1.5. Colonies which gave the expected PCR product size (719 bp+106 bp due to the multiple cloning site or MCS) were grown up in 5 ml LB containing ampicillin (100 μg/ml), overnight with shaking at 220 rpm, at 37° C. Miniprep plasmid DNA was prepared from 5 ml cultures and sequenced with T3 and T7 primers as described in section 1.6. The sequence of one of the resulting clones and the corresponding plasmid map (pCR4 blunt TOPO-INSP002V) are shown in FIGS. 12 and 13 respectively. Translation of the cloned sequence indicates that INSP002V contains a 2 amino acid deletion (ΔV107 and ΔQ108) and a single amino acid substitution (F110L) compared to the predicted INSP002 sequence. Alignment of the nucleotide and amino acid sequences for the INSP002 prediction and INSP002V are shown in FIG. 14.

When compared to the INSP002 prediction, the splice junction between exons 1 and 2 uses a 6 bp upstream donor site. This results in the deletion of two amino acids (ValGlu). The splice acceptor used is the same as that used by the INSP002 prediction, however there are two sequencing errors after the acceptor. This results in an amino acid substitution (Phe→Leu).

### Example 5

### Construction of a Plasmid for the Expression of INSP002V in HEK293/EBNA Cells

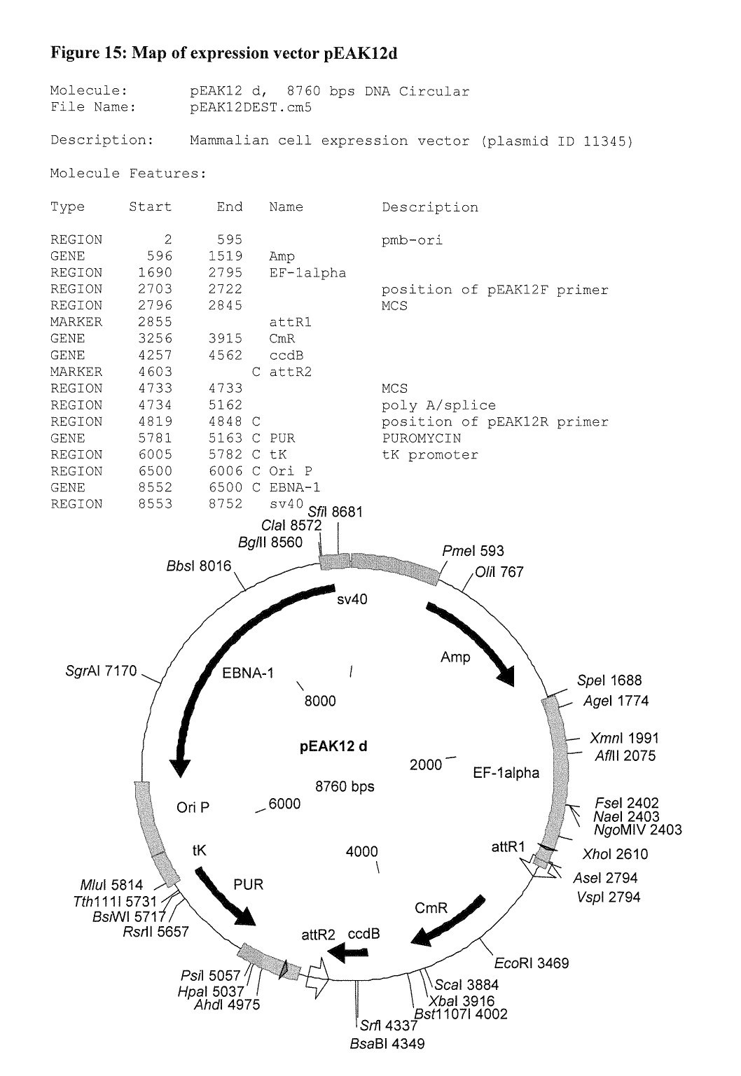

A pCR4 blunt-TOPO clone containing the full coding sequence (ORF) of INSP002V identified by DNA sequencing (FIG. 13) was then used to subclone the insert into the mammalian cell expression vector pEAK12d (FIG. 15) using the Gateways cloning methodology (Invitrogen).

i) Generation of Gateway Compatible INSP002 ORF Fused to an in Frame 6HIS Tag Sequence.

The first stage of the Gateway cloning process involves a two step PCR reaction which generates the ORF of INSP002V flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction (in a final volume of 50 μl) contains: 25 ng of pCR4 blunt TOPO-INSP002V (plasmid 13075 and FIG. 13), 1.5 μl dNTPs (10 mM), 5 μl of 10×Pfx polymerase buffer, 1 μl MgSO4 (50 mM), 0.5 μl each of gene specific primer (100 μM) (INSP002V-EX1 and INSP002V EX2) and 0.5 μl Platinum Pfx DNA polymerase (Invitrogen). The PCR reaction was performed using an initial denaturing step of 95° C. for 2 min, followed by 12 cycles of 94° C., 15 sec and 68° C. for 30 sec. PCR products were purified directly from the reaction mixture using the Wizard PCR prep DNA purification system (Promega) according to the manufacturer's instructions. The second PCR reaction (in a final volume of 50 μl) contained 10 μl purified PCR product, 1.5 μl dNTPs (10 mM), 1 μl MgSO4 (50 mM), 5 μl of 10×Platinum Pfx polymerase buffer, 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 45° C., 30 sec and 68° C. for 3.5 min; 25 cycles of 94° C., 15 sec; 55° C., 30 sec and 68° C., 3.5 min. PCR products were purified as described above.

ii) Subcloning of Gateway Compatible INSP002V ORF into Gateway Entry Vector pDONR201 and Expression Vector pEAK12d

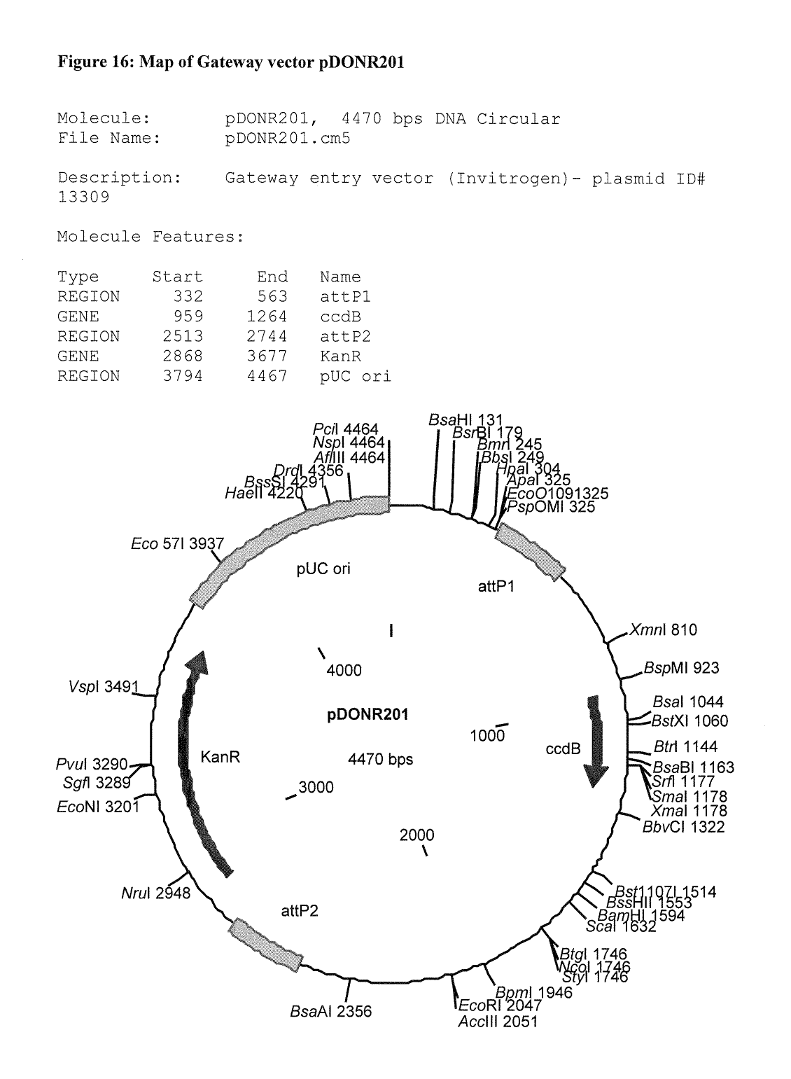

The second stage of the Gateway cloning process involves subcloning of the Gateway modified PCR product into the Gateway entry vector pDONR201 (Invitrogen, FIG. 16) as follows: 5 μl of purified PCR product is incubated with 1.5 μl pDONR201 vector (0.1 μg/μl), 2 μl BP buffer and 1.5 μl of BP clonase enzyme mix (Invitrogen) at RT for 1 h. The reaction was stopped by addition of proteinase K (2 μg) and incubated at 37° C. for a further 10 min. An aliquot of this reaction (2 μl) was transformed into E. coli DH10B cells by electroporation using a Biorad Gene Pulser. Transformants were plated on LB-kanamycin plates. Plasmid mini-prep DNA was prepared from 1-4 of the resultant colonies using Wizard Plus SV Minipreps kit (Promega), and 1.5 μl of the plasmid eluate was then used in a recombination reaction containing 1.5 μl pEAK12d vector (FIG. 9) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation.

Clones containing the correct insert were identified by performing colony PCR as described above except that pEAK12d primers (pEAK12d F and pEAK12d R) were used for the PCR Plasmid mini prep DNA was isolated from clones containing the correct insert using a Qiaprep Turbo 9600 robotic system (Qiagen) or manually using a Wizard Plus SV minipreps kit (Promega) and sequence verified using the pEAK12d F and pEAK12d R primers.

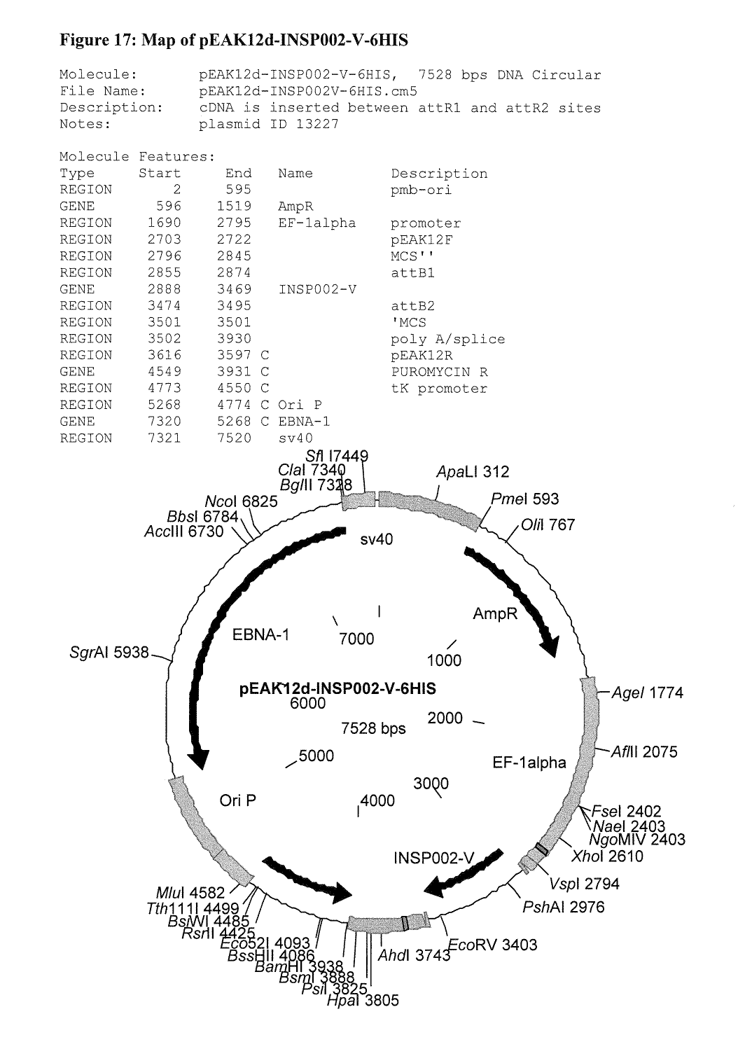

CsCl gradient purified maxi-prep DNA of plasmid pEAK12d-INSP002V-6HIS (plasmid ID number 13227, FIG. 17) was prepared from a 500 ml culture of sequence verified clones (Sambrook J. et al., in Molecular Cloning, A Laboratory Manual, 2^{nd} edition, 1989, Cold Spring Harbor Laboratory Press), resuspended at a concentration of 1 μg/ml in sterile water and stored at −20 C.

iii) Construction of Expression Vector pEAK12d

The vector pEAK12d is a Gateway Cloning System compatible version of the mammalian cell expression vector pEAK12 (purchased from Edge Biosystems) in which the cDNA of interest is expressed under the control of the human EF1α promoter. pEAK12d was generated as described below:

pEAK12 was digested with restriction enzymes HindIII and NotI, made blunt ended with Klenow (New England Biolabs) and dephosphorylated using calf-intestinal alkaline phosphatase (Roche). After dephosphorylation, the vector was ligated to the blunt ended Gateway reading frame cassette C (Gateway vector conversion system, Invitrogen cat no. 11828-019) which contains AttR recombination sites flanking the ccdB gene and chloramphenicol resistance, and transformed into E. coli DB3.1 cells (which allow propagation of vectors containing the ccdB gene). Mini prep DNA was isolated from several of the resultant colonies using a Wizard Plus SV Minipreps kit (Promega) and digested with AseI/EcoRI to identify clones yielding a 670 bp fragment, indicating that the cassette had been inserted in the correct orientation. The resultant plasmid was called pEAK12d (FIG. 15).

### Example 6

### Expression in Mammalian Cells and Purification of the INSP002SV-6His-V1 (Plasmid #13227)

Human Embryonic Kidney 293 cells expressing the Epstein-Barr virus Nuclear Antigen (HEK293-EBNA, Invitrogen) were maintained in suspension in Ex-cell VPRO serum-free medium (seed stock, maintenance medium, JRH). Sixteen to 20 hours prior to transfection (Day-1), cells were seeded in 2×T225 flasks (50 ml per flask in DMEM/F12 (1:1) containing 2% FBS seeding medium (JRH) at a density of 2×10⁵ cells/ml). The next day (transfection day 0) the transfection took place by using the JetPEI™ reagent (2 μl/μg of plasmid DNA, PolyPlus-transfection). For each flask, 113 μg of cDNA (number #13227) was co-transfected with 2.3 μg of GFP (fluorescent reporter gene). The transfection mix was then added to the 2×T225 flasks and incubated at 37° C. (5% CO₂) for 6 days. In order to increase our chances to get more material, we repeated this procedure into two extra flasks such as to generate 200 ml total. Confirmation of positive transfection was done by qualitative fluorescence examination at day 1 and day 6 (Axiovert 10 Zeiss).

On day 6 (harvest day), supernatants (200 ml) from the four flasks were pooled and centrifuged (4° C., 400 g) and placed into a pot bearing a unique identifier.

One aliquot (500 ul) was kept for QC of the 6His-tagged protein (internal bioprocessing QC).

Purification Process

The 200 ml culture medium sample containing the recombinant protein with a C-terminal 6His tag was diluted to a final volume of 400 ml with cold buffer A (50 mM NaH₂PO₄; 600 mM NaCl; 8.7% (w/v) glycerol, pH 7.5). The sample was filtered through a 0.22 μm sterile filter (Millipore, 500 ml filter unit) and kept at 4° C. in a 500 ml sterile square media bottle (Nalgene).

The purification was performed at 4° C. on the VISION workstation (Applied Biosystems) connected to an automatic sample loader (Labomatic). The purification procedure was composed of two sequential steps, metal affinity chromatography on a Poros 20 MC (Applied Biosystems) column charged with Ni ions (4.6×50 mm, 0.83 ml), followed by gel filtration on a Sephadex G-25 medium (Amersham Pharmacia) column (1.0×10 cm).

For the first chromatography step the metal affinity column was regenerated with 30 column volumes of EDTA solution (100 mM EDTA; 1 M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample was charged onto the Ni metal affinity column in batches of 200 ml. The Labomatic sample loader transferred 200 ml of the sample into a 200 ml sample loop and this sample was subsequently charged onto the Ni metal affinity column at a flow rate of 10 ml/min. The transfer and charging procedure was repeated once to load the 400 ml sample onto the column. At the end of the charging procedure the column was washed with 12 column volumes of buffer A, followed by 28 column volumes of buffer A containing 20 mM imidazole. During the 20 mM imidazole wash loosely attached contaminating proteins were elution of the column. The recombinant His-tagged protein was finally eluted with 10 column volumes of buffer B at a flow rate of 2 ml/min, and the eluted protein was collected in a 1.6 ml fraction.

For the second chromatography step, the Sephadex G-25 gel-filtration column was regenerated with 2 ml of buffer D (1.137 M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column was automatically, through the integrated sample loader on the VISION, loaded onto the Sephadex G-25 column and the protein was eluted with buffer C at a flow rate of 2 ml/min. The desalted sample was recovered in a 2.2 ml fraction. The fraction was filtered through a 0.22 μm sterile centrifugation filter (Millipore), aliquoted, frozen and stored at −80 C. An aliquot of the sample was analyzed on SDS-PAGE (4-12% NuPAGE gel; Novex) Western blot with anti-His antibodies.

Following the electrophoresis the proteins were electrotransferred from the gel to a nitrocellulose membrane at 290 mA for 1 hour at 4° C. The membrane was blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2 ug/ml each; Santa Cruz) in 2.5% milk powder in buffer E overnight at 4° C. After further 1 hour incubation at room temperature, the membrane was washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane was developed with the ECL kit (Amersham Pharmacia) for 1 min. The membrane was subsequently exposed to a Hyperfilm (Amersham Pharmacia), the film developed and the western blot image visually analyzed.

### Example 7

### Cloning of Full Length INSP002 from Heart

The full length coding sequence of INSP002 was cloned from heart cDNA as follows:

i) Generation of Heart cDNA Template

Human heart total RNA from was purchased from Clontech. The quality and concentration of the RNA was analysed using an Agilent 2100 Bioanalyzer.

For cDNA synthesis the reaction mixture contained: 1 μl oligo (dT)₁₅ primer (500 μg/ml, Promega cat. no. C 1101), 2 μg total RNA, 1 μl dNTPs (10 mM) in a volume of 12 μl. The mixture was heated to 65° C. for 5 min and then chilled on ice. The following reagents were then added: 4 μl 5×first strand buffer, 2 μl DTT (0.1 M), 1 μl RNAseOut recombinant ribonuclease inhibitor (40 units/μl, Promega, cat. no. N 2511) and incubated at 42° C. for 2 min before addition of 1 μl (200 units) of Superscript II (Invitrogen cat. no. 18064-014). The mixture was incubated at 42° C. for 50 min and then heated at 70° C. for 15 min. To remove the RNA template, 1 μl (2 units) of E. coli RNase H (Invitrogen cat. no.18021-014) was added and the reaction mixture further incubated at 37° C. for 20 min. The final reaction mix was diluted to 200 μl with sterile water and stored at −80 C.

ii) Cloning of the Full Length Coding Sequence of INSP002 by PCR

The full length coding sequence of INSP002 was cloned from human heart cDNA by PCR in a 50 μl PCR reaction mixture containing 5 μl heart cDNA, 5 μl 10×Pfx buffer, 1.5 μl dNTPs (10 mM), 1 μl MgSO4 (50 mM), 1.5 μl gene specific forward primer INSP002-FL-F (10 μM), 1.5 μl gene specific reverse primer INSP002-FL-R (10 μM) and 0.5 μl Platinum Pfx DNA polymerase (Invitrogen). The cycling conditions were 1 cycle of 94° C., 4 min; 35 cycles of 94° C., 15 sec; 55° C., 30 s; 68° C., 1 min; 1 cycle of 68° C., 10 min followed by a holding cycle at 4° C.

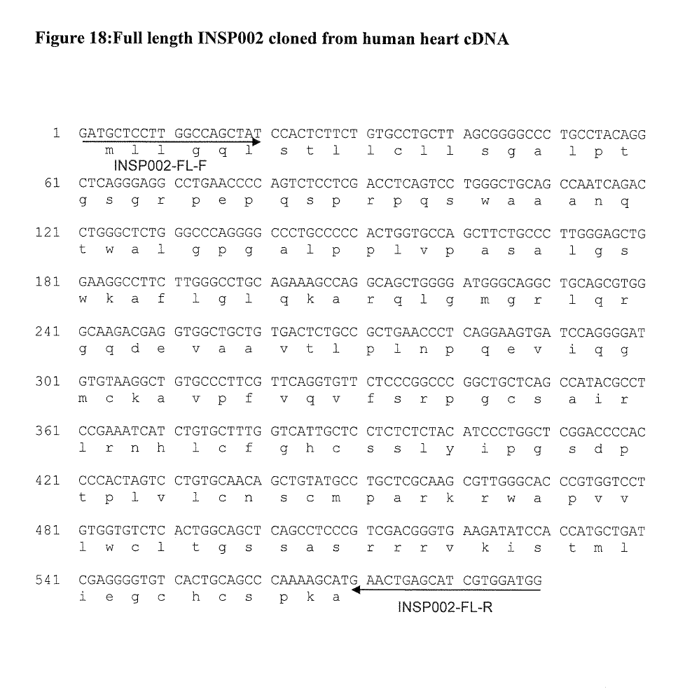

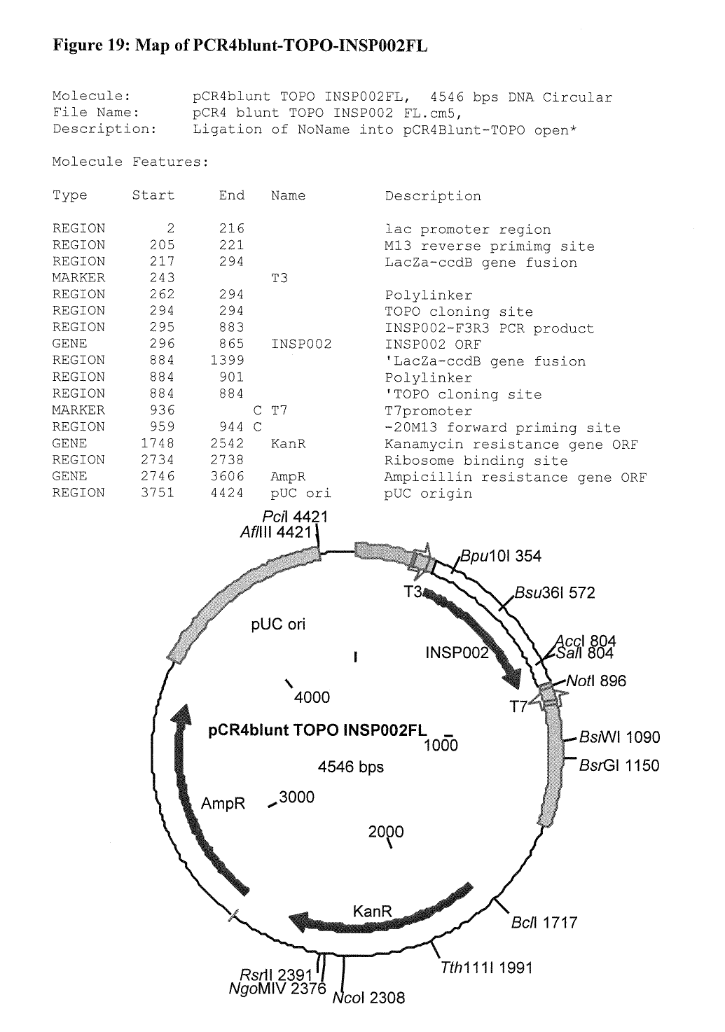

The amplification products were visualized on 0.8% agarose gels in 1×TAE buffer (Invitrogen) and PCR products migrating at the predicted molecular mass (589 bp) were purified from the gel using the Qiagen MinElute Gel Extraction kit (Qiagen). PCR products were eluted in 50 μl of 10 mM Tris-HCl pH 8.5 and subcloned into pCR4 blunt TOPO vector as described previously (section 1.4) Several ampicilin resistant colonies were subjected to colony PCR as described in section 1.5. Colonies containing the correct size insert (589 bp+106 bp due to the MCS) were grown up overnight at 37° C. in 5 ml L-Broth (LB) containing ampicillin (100 μg/ml), with shaking at 220 rpm at 37° C. Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions and 200-500 ng of mini-prep DNA was sequenced as described in section 1.6 with T3 and 17 primers (Table III). The cloned sequence is given in FIG. 18. The map of the resultant plasmid, pCR4-blunt TOPO-INSP002FL (plasmid ID. No. 13514) is shown in FIG. 19.

### Example 8

### INSP002 Inhibits the Action of TGF Beta in Human Breast Cancer Cells

A plasmid vector was constructed containing cDNA encoding full-length INSP002 without the signal peptide (SEQ ID NO:16). Plasmids containing cDNA encoding N-terminal (aa1-68 without signal peptide) or C-terminal (aa 69-719) fragments of INSP002 fused to an Fc domain (SEQ ID NO:18 and 20) and plasmids containing cDNA encoding N-terminal fragment of INSP002 fused to an hCGbeta subunit (SEQ ID NO:24) or the modified hCGalpha subunit alpha des88-92 (SEQ ID NO:22) were also constructed.

The plasmid encoding the full-length INSP002 fused to Fc was used to expresss an INSP002-Fc polypeptide in HEK 293 cells or as a His-tagged molecule in E. coli. The preparation of constructs containing the His-tagged INSP002-Fc cDNA (SEQ ID NO:26) and the INSP002-Fc cDNA in a form suitable for expression in detail below.

In the case of HEK 293 expression system, HEK293 cells were transfected with pEAK12d vectors encoding the INSP002-FC protein using standard transfection protocols. Cells were grown in DMEM high glucose medium containing 10% FBS and 1% glutamine. Cells were harvested 72 hours after transfection and the protein was purified using either Protein A column or conventional chromatography and tested in a cell-based assay. In the case of E. coli expression system, the protein was refolded from inclusion bodies.

The cell-based assay was developed on an existing animal model of breast cancer metastatic disease. Briefly, MDA-MB-231 cells were treated with TGFbeta in the presence or absence of different amount of INSP002-Fc for 48 hours. The conditioned media were collected and assayed for the amounts of IL-11 produced by these cells in response to TGFbeta. IL-11 ELISA kit was purchased from R&D Systems. Further details of the assay are provided below. Anti-TGFb antibodies (R&D Systems) and OPG-Fc (Serono) were used as positive and negative controls, respectively.

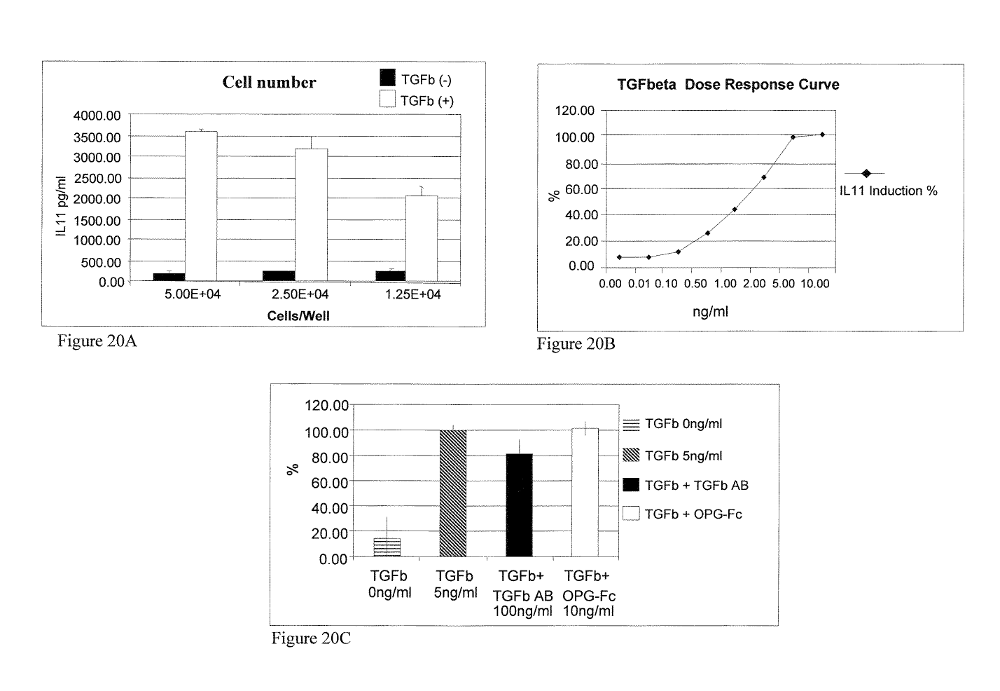

FIG. 20 shows the effect of 5 ng/ml TGFb on IL-11 production by MDA-MB-231 cells in the absence of INSP002-Fc polypeptide. MDA-MB-231 cells were plated at a density of 12500, 25000 or 50000 cells per well in the presence of absence of TGFbeta and the amount of IL-11 produced by the cells was measured after 48 hours. As shown FIG. 20A, a significant increase in IL-11 production was seen at all three densities but the optimum effect was noted at 50,000 cells per well. FIG. 20B shows the effect of different concentrations of TGF-beta on IL-11 production by cells plated at a density of 50,000 cells/well. A 5 ng/ml dose of TGFbeta was chosen for further experiments because of the robustness of the assay at this concentration. FIG. 20C shows the results of the same IL-11 assay in the present or absence of TGFbeta and in the presence or absence of positive (TGFbeta monoclonal antibodies) and negative (OPG-Fc; osteoprotegerin fused with IgG1 Fc) controls.

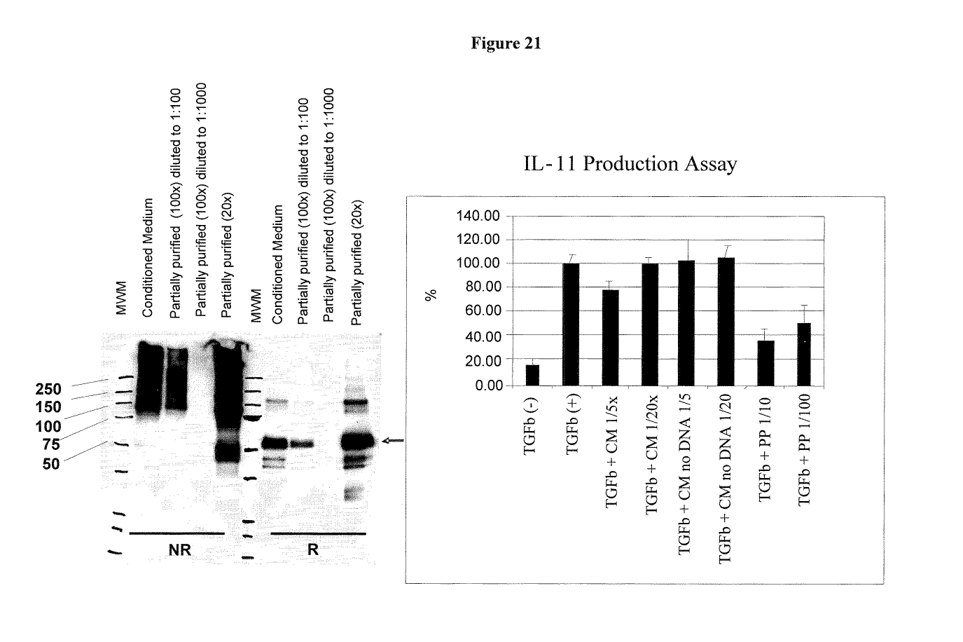

The effect of the partially purified INSP002-Fc polypeptide on IL-11 production was tested (FIG. 21). MDA-MB-231 cells were incubated for 48 hours in the presence or absence of 5 ng/ml TGFbeta as controls. Cells were also incubated for 8 hours in the presence of 5 ng/ml TGFbeta and: a) non-purified conditioned media (TGFb+CM), b) conditioned media from a cell without transfection (TGFb+CM no DNA) or c) partially purified polypeptide (TGFb+PP). The conditioned media were added at dilutions of ⅕ or 1/20 and the partially purified polypeptide was added at dilutions of 1/10 or 1/100. As shown in FIG. 21, the presence of partially purified polypeptide reduced IL-11 production from 100% to around 40%.

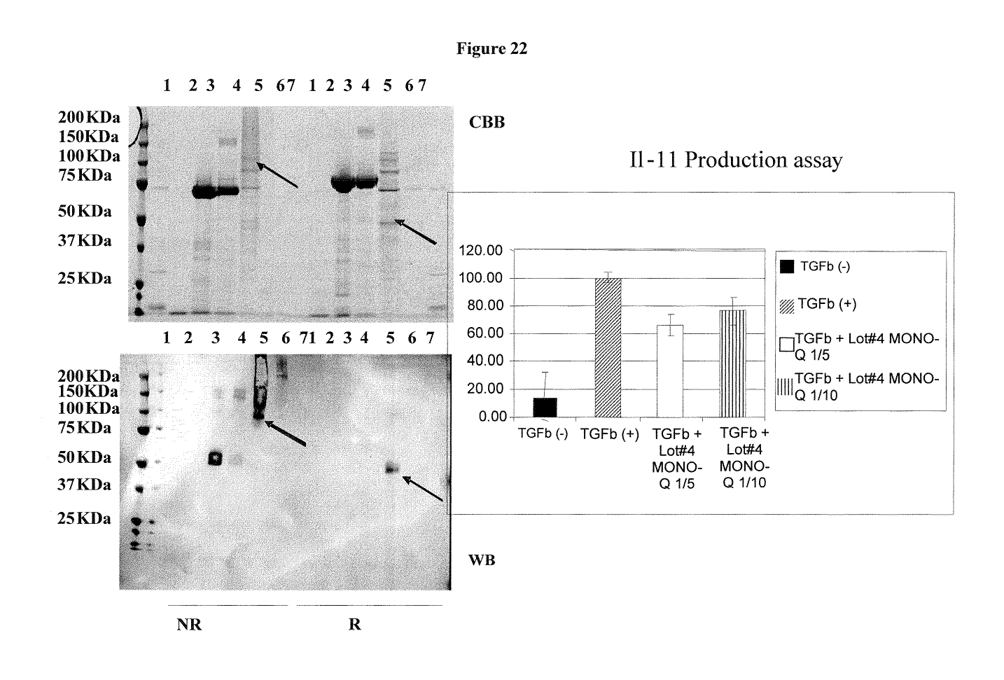

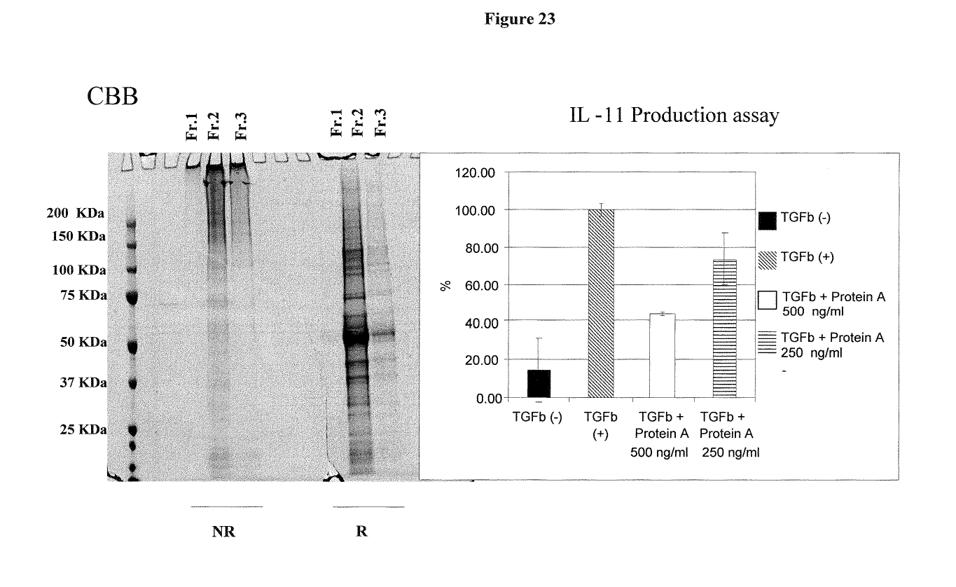

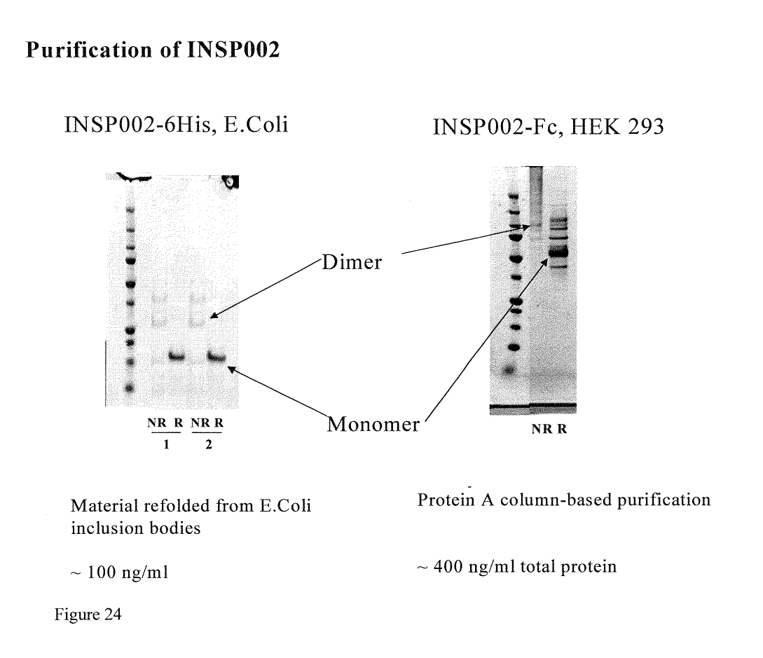

The INSP002-Fc polypeptide expressed from HEK293 cells was further purified by conventional chromatography or Protein A column. The INSP002 polypeptide purified from the Protein A column was estimated to be present in a concentration of 400 ng/ml (FIG. 22). The ability of the polypeptide purified by both methods to inhibit IL-11 production induced by 5 ng/ml of TGFb was tested (FIG. 23 and FIG. 24). As shown in FIG. 24, 500 ng/ml of INSP002-Fc polypeptide reduced TGFbeta mediated production by MDA-MB-231 cells from 100% in the presence of 5 ng/ml TGFbeta to around 40% and 250 ng/ml of INSP002-Fc polypeptide reduced IL-11 production to around 75%.

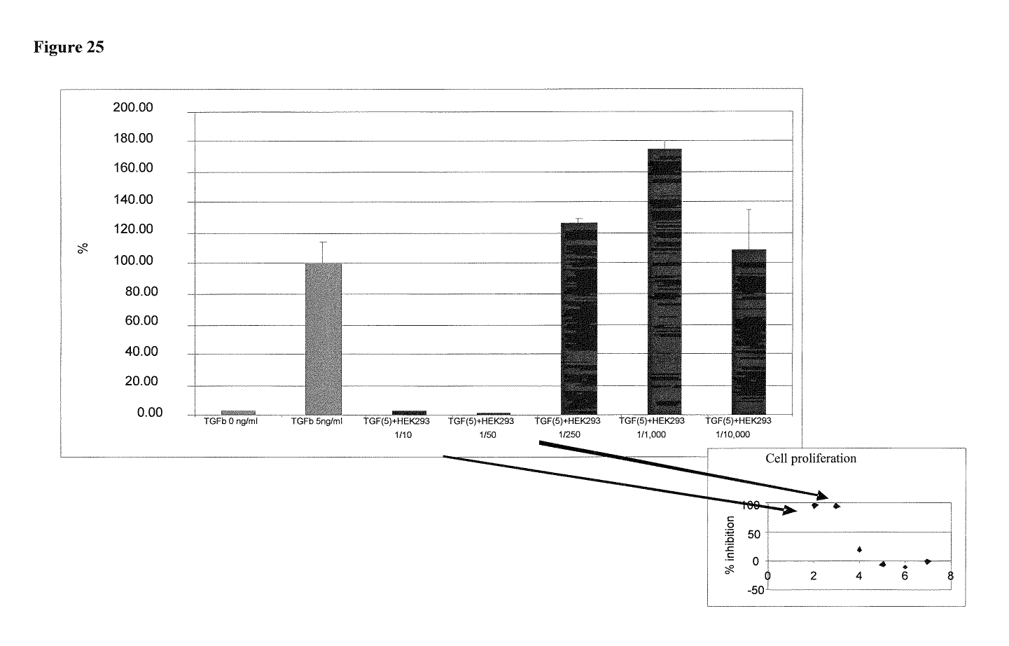

FIG. 25 shows a further experiment in which different concentrations of the INSP002-Fc polypeptide purified by Protein A chromatography (dilutions of 1/10, 1/50, 1/250, 1/1000 and 1/10000) were tested for the ability to reduce IL-11 production by 5 ng/ml TGFb. At high concentrations (dilutions of 1/10 and 1/50), the purified INSP002-Fc polypeptide reduced IL-11 production to levels in the absence of TGFb. The exact concentrations of the purified polypeptide used in the dilutions were not determined but, on the basis that the original polypeptide was estimated to be present at a concentraion of 400 ng/ml (see FIG. 22), it is estimated that the 1/10 dilution contained around 40 ng/ml of the polypeptide and the 1/50 dilution contained around 8 ng/ml of polypeptide. At lower concentrations, the polypeptide did not reduce IL-11 production. At a dilution of 1/1000, IL-11 production was increased by the presence of the polypeptide, an anomalous result which may be due to a contaminant.

The effect of different concentrations of the polypeptide on cell proliferation was also measured. As shown in the lower panel of FIG. 25, the presence of high concentrations of the polypeptide (dilutions of 1/10 and 1/50) inhibited cell proliferation completely (100%). No cells were presnt after the 48 hour incubation period with the polypeptide, suggesting that the presence of high concentrations of the INSP002-Fc polypeptide kills the breast cancer cells. The reduction in IL-11 levels at high concentrations of the polypeptide may therefore be due to the absence of cells.

In summary, these results indicate that INSP002-Fc reduces TGFb-mediated production of IL-11 by metastatic breast adenocarcinoma MDA-MB-231 cells. Down-regulation of IL-11 production by INSP002 was shown in the presence of high amount of protein. At these concentrations, INSP002 was also found to inhibits cell proliferation and, at very high doses, INSP002 was found to kill the cancerous cells.

Materials and Methods

TGFβ Induction of Cells on 48-Well Plate and Collection of 48-Hour Conditioned Media
1. 50 000 MDA-MB-231 (ATCC) were plated into 48-well plates
2. 0.5 ml of Leibovitz media (Gibco) with 10% fetal calf serum added to the wells
3. 24 hours later the cells were washed with 1 ml warm PBS and 0.5 ml of media with 0.5-5.0 ng/ml of TGFb1 (no serum) added
4. The plates were incubated for 48 hours in CO₂ incubator
5. The media from the wells were collected for IL-11 production
6. IL-11 ELISA is performed according to the protocol from R&D Systems

Generation of Full Length INSP002-Fc and INSP002 Constructs

1. Plasmid 6His-Full Length INSP002-Fc in pET21d, MB0245

Materials

Insert; INSP002cDNA from plasmid Full Length INSP002-Fc in DalphaDEST at 20 ng/ul Vector; pET21d Novagen 69743-3

Method

1. PCR; Addition of 5′-NcoI/3′-XhoI Sites and N-Term 6His Tag.

Forward Primer
Nco-6H-002-Final F; gaattcccatggctcaccatcaccatcacc
ataggcctgaaccccagtct
Reverse Primer
002-XhoI-R; gaattcctcgagctatgcttttgggctgcag

Template; Full Length INSP002-Fc in DalphaDEST 20 ng/ul

| | |
|---|---|
| Template | 1 ul |
| 10× Buffer | 5 ul |
| 10 mM dNTP | 2 ul |
| 25 mM F-primer | 2 ul |
| 25 mM R-primer | 2 ul |
| 25 mM MgSo4 | 8 ul |
| Ultra Pfu Turbo | 1 ul (Stratagene) |
| DW | 29 |
| Total | 50 ul |

| | | |
|---|---|---|
| Cycles; | 95 C. 2 min. | |
| | 95 C. 30 sec. | |
| | 65 C. 30 sec. | |
| | 72 C. 2 min. | |
| | 4 C. Hold | Repeat Cycle 2-4 40×'s |

2. Purification of the PCR Product

PCR cleaning Kit by Qiagen

Dissolved in 50 ul DW

3. Restriction Digestion by NcoI/XhoI (NEB)

| | |
|---|---|
| Purified PCR product | 8 ul |
| NcoI | 1 ul |
| XhoI | 1 ul |
| 10× NEB2 Buffer | 2 ul |
| 10× BSA buffer | 2 ul |
| DW | 6 ul |
| Total | 20 ul |

at 37 C, 1 hr

4. Gel Purification

Gel Purification Kit by Qiagen

Dissolved in 30 ul DW

5. Ligation

| | |
|---|---|
| Insert | 3 ul |
| Vector | 0.5 ul |
| T4 DNA ligase | 1 ul |
| 10× ligase buffer | 1 ul |
| DW | 4.5 ul |
| Total | 10 ul |

at 16 C O/N

6. Transformation

5 ul of Ligation mix was transformed into 100 ul of JM109 (Promega).

7. Miniprep (Qiagen)˜Restriction Digestion Analysis by NcoI/XhoI

8. Sequencing to Verify cDNA

ABI Genetic Analyzer 3100

2. Plasmid Full Length INSP002-Fc in pREST-A, MB0245

Materials

Insert; INSP002cDNA from plasmid Full Length INSP002-Fc in DalphaDEST at 20 ng/ul

Vector; pRSET-A O

Method

1. PCR; Addition of 5′-NdeI/3′-XhoI Sites

Forward Primer
Nde-002-F; gggaaattccatatgaggcctgaaccccagtct
Reverse Primer
002-XhoI-R; gaattcctcgagctatgcttttgggctgcag

Template; Full Length INSP002-Fc in DalphaDEST—Miniprep diluted 1:10

| | |
|---|---|
| Template | 2 ul |
| AB gene Master Mix | 25 ul |
| 25 mM F-primer | 1 ul |
| 25 mM R-primer | 1 ul |
| DW | 21 |
| Total | 50 ul |

| | | | |
|---|---|---|---|
| Cycles; | 1. | 94 C. 2 min. | |
| | 2. | 94 C. 30 sec. | |
| | 3. | 65 C. 30 sec. | |
| | 4. | 68 C. 2 min. | |
| | 5. | 68 C. 7 min. | |
| | 6. | 4 C. Hold | Repeat Cylce 2-4 30×'s |

2. Purification of the PCR Product

PCR cleaning Kit by Qiagen

Dissolved in 50 ul DW

3. Restriction Digestion by NcoI O/XhoI (NEB)

| | |
|---|---|
| Purified PCR product | 30 ul |
| NdeI | 4 ul |
| XhoI | 1 ul |
| 10× H buffer | 5 ul |
| 10× BSA buffer | 5 ul |
| DW | 5 ul |
| Total | 50 ul |

at 37 C, 1 hr

4. Gel Purification

Gel Purification Kit by Qiagen

Dissolved in 30 ul DW

5. Ligation

| | |
|---|---|
| Insert | 1 ul |
| Vector | 1 ul |
| T4 DNA ligase | 1 ul |
| 10× ligase buffer | 1 ul |
| DW | 6 ul |
| Total | 10 ul |

at 16 C for 11 days

6. Transformation

5 ul of Ligation mix was transformed into 100 ul of JM109 (Promega).

7. Miniprep (Qiagen)

8. Sequencing to Verify cDNA

ABI Genetic Analyzer 3100

TABLE I
Human cDNA libraries

| Library | Tissue/cell source | Vector | Host strain | Supplier | Cat. no. |
|---|---|---|---|---|---|
| 1 | Human fetal brain | Zap II | XL1-Blue MRF′ | Stratagene | 936206 |
| 2 | Human ovary | GT10 | LE392 | Clontech | HL1098a |
| 3 | Human pituitary | GT10 | LE392 | Clontech | HL1097a |
| 4 | Human placenta | GT11 | LE392 | Clontech | HL1075b |
| 5 | Human testis | GT11 | LE392 | Clontech | HL1010b |
| 6 | Human sustanta nigra | GT10 | LE392 | in house | |
| 7 | Human fetal brain | GT10 | LE392 | in house | |
| 8 | Human cortex brain | GT10 | LE392 | in house | |
| 9 | Human colon | GT10 | LE392 | Clontech | HL1034a |
| 10 | Human fetal brain | GT10 | LE392 | Clontech | HL1065a |
| 11 | Human fetal lung | GT10 | LE392 | Clontech | HL1072a |
| 12 | Human fetal kidney | GT10 | LE392 | Clontech | HL1071a |
| 13 | Human fetal liver | GT10 | LE392 | Clontech | HL1064a |
| 14 | Human bone marrow | GT10 | LE392 | Clontech | HL1058a |
| 15 | Human peripheral blood monocytes | GT10 | LE392 | Clontech | HL1050a |
| 16 | Human placenta | GT10 | LE392 | in house | |
| 17 | Human SHSYSY | GT10 | LE392 | in house | |
| 18 | Human U373 cell line | GT10 | LE392 | in house | |
| 19 | Human CFPoc-1 cell line | Uni Zap | XL1-Blue MRF′ | Stratagene | 936206 |
| 20 | Human retina | GT10 | LE392 | Clontech | HL1132a |
| 21 | Human urinary bladder | GT10 | LE392 | in house | |
| 22 | Human platelets | Uni Zap | XL1-Blue MRF′ | in house | |
| 23 | Human neuroblastoma Kan + TS | GT10 | LE392 | in house | |
| 24 | Human bronchial smooth muscle | GT10 | LE392 | in house | |
| 25 | Human bronchial smooth muscle | GT10 | LE392 | in house | |
| 26 | Human Thymus | GT10 | LE392 | Clontech | HL1127a |
| 27 | Human spleen 5′ stretch | GT11 | LE392 | Clontech | HL1134b |
| 28 | Human peripherical blood monocytes | GT10 | LE392 | Clontech | HL1050a |
| 29 | Human testis | GT10 | LE392 | Clontech | HL1065a |
| 30 | Human fetal brain | GT10 | LE392 | Clontech | HL1065a |
| 31 | Human substancia Nigra | GT10 | LE392 | Clontech | HL1093a |
| 32 | Human placenta#11 | GT11 | LE392 | Clontech | HL1075b |
| 33 | Human Fetal brain | GT10 | LE392 | Clontech | custom |
| 34 | Human placenta #59 | GT10 | LE392 | Clontech | HL5014a |
| 35 | Human pituirary | GT10 | LE392 | Clontech | HL1097a |
| 36 | Human pancreas #63 | Uni Zap XR | XL1-Blue MRF′ | Stratagene | 937208 |
| 37 | Human placenta #19 | GT11 | LE392 | Clontech | HL1008 |
| 38 | Human liver 5′ strech | GT11 | LE392 | Clontech | HL1115b |
| 39 | Human uterus | Zap-CMV XR | XL1-Blue MRF′ | Stratagene | 980207 |
| 40 | Human kidney large-insert cDNA library | TriplEx2 | XL1-Blue | Clontech | HL5507u |

TABLE II
INSP002 Cloning primers

| Primer | Sequence (5′-3′) |
|---|---|
| INSP002-CP1 | CTC AGC CAT ACG CCT CCG AA |
| INSP002-CP2 | GCT GAG CTG CCA GTG AGA CA |
| INSP002V-5′-F | ACC TGG AAG GAA GCG ACT GCA CTG |
| | A |
| INSP002V-5′-R | GCA GCC GGG CCG GGA GAG AAC GAA |
| | GGG CAC AGC CTT A |
| INSP002V-3′-F | AGG CTG TGC CCT TCG TTC TCT CCC |
| | GGC CCG GCT GCT C |
| INSP002V-3′-R | ACT CCA GGA CGG GCA CTG TGT CTA |
| | C |
| INSPO02V-5′nest-F | GTC GAC TGC TAG TGA CCT TGA G |
| INSPO02V-3′nest-R | ACA TCA TCC AGG TCC ACG TCT T |

TABLE III
Primers for INSP002 subcloning and sequencing

| Primer | Sequence (5′-3′) |
|---|---|
| SP6 | ATT TAG GTG ACA CTA TAG |
| T7 | TAA TAC GAC TCA CTA TAG GG |
| T3 | ATT AAC CCT CAC TAA AGG GA |
| M13F | TGT AAA ACG ACG GCC AGT |
| pEAK12-F | GCC AGC TTG GCA CTT GAT GT |
| pEAK12-R | GAT GGA GGT GGA CGT GTC AG |
| INSP002V-EX1 | AA GCA GGC TTC GCC ACC ATG CTC CTT |
| | GGC CAG CTA TC |
| INSP002V-EX2 | GTG ATG GTG ATG GTG TGC TTT TGG GCT |
| | GCA GTG AC |
| GCP Forward | G GGG ACA AGT TTG TAC AAA AAA GCA GGC |
| | TTC GCC ACC |
| GCP Reverse | GGG GAC CAC TTT GTA CAA GAA AGC TGG |
| | GTT TCA ATG GTG ATG GTG ATG GTG |

TABLE IV
PCR primers for cloning of full length INSP002

| Primer | Sequence (5′-3′) |
|---|---|
| INSP002-FL-F | GAT GCT CCT TGG CCA GCT AT |
| INSP002-FL-R | CCA TCC ACG ATG CTC AGT TC |

Underlined sequence = Kozak sequence
Bold = Stop codon
Italic sequence = His tag

1-18. (canceled)

19. A method of treating a cancer or a disorder associated with cancer comprising administering to a patient in need thereof:
a) a polypeptide which:
i) comprises or consists of SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27;
ii) is a fragment of a polypeptide comprising or consisting of SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27 and has the function of a member of the DAN subfamily; or
iii) is a variant of (i) or (ii), said variant having at least 85% identity to a polypeptide of (i) or (ii) and has the ability to inhibit proliferation of breast cancer cells; or
b) a purified nucleic acid molecule encoding a polypeptide according to part a).

20. The method according to claim 19, wherein said disease is metatstatic cancer.

21. The method according to claim 20, wherein said disease is breast cancer.

22. The method according to claim 19, wherein said method reduces levels of TGF beta-induced IL-11.

23. The method according to claim 19, wherein the proliferation of tumor cells is inhibited.

24. The method according to claim 19, wherein said cancer is treated by killing tumor cells.

25. The method according to claim 19, wherein said polypeptide is administered at a concentration of between 5 and 50 ng/ml.

26. The method according to claim 19, wherein said polypeptide is a variant of a polypeptide comprising SEQ ID NO:6, SEQ ID NO:8, SEQ ID NO:14, SEQ ID NO:17, SEQ ID NO:19, SEQ ID NO:21, SEQ ID NO:23, SEQ ID NO:25, SEQ ID NO:27 or fragments thereof and has the ability to inhibit proliferation of breast cancer cells.

27. The method according to claim 19, wherein said polypeptide is a variant of a polypeptide consisting of SEQ ID NO:6, SEQ ID NO:8, SEQ ID NO:14, SEQ ID NO:17, SEQ ID NO:19, SEQ ID NO:21, SEQ ID NO:23, SEQ ID NO:25, SEQ ID NO:27 or fragments thereof and has the ability to inhibit proliferation of breast cancer cells.

28. The method according to claim 19, wherein said polypeptide is a fragment of SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27 and has the ability to inhibit proliferation of breast cancer cells.

29. The method according to claim 19, wherein the purified nucleic acid molecule comprises SEQ ID NO:5, SEQ ID NO:9, SEQ ID NO:11, SEQ ID NO:17, SEQ ID NO:19, SEQ ID NO:21, SEQ ID NO:23 and SEQ ID NO:25, SEQ ID NO:27 or encodes a polypeptide comprising SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27 or is a fragment thereof.

30. The method according to claim 19, wherein the purified nucleic acid molecule comprises SEQ ID NO:5, SEQ ID NO:9, SEQ ID NO:11, SEQ ID NO:17, SEQ ID NO:19, SEQ ID NO:21, SEQ ID NO:23 and SEQ ID NO:25, SEQ ID NO:27 or encodes a polypeptide comprising SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27.

31. The method according to claim 19, wherein the purified nucleic acid molecule comprises a fragment of SEQ ID NO:5, SEQ ID NO:9, SEQ ID NO:11, SEQ ID NO:17, SEQ ID NO:19, SEQ ID NO:21, SEQ ID NO:23 and SEQ ID NO:25, SEQ ID NO:27 or encodes a polypeptide fragment of SEQ ID NO:6, SEQ ID NO:8, SEQ ID NO:14, SEQ ID NO:17, SEQ ID NO:19, SEQ ID NO:21, SEQ ID NO:23, SEQ ID NO:25 or SEQ ID NO:27 that inhibits the proliferation of breast cancer cells.


SEQUENCE LISTING (68 sequences)

SEQ ID NO: 1 - DNA, 475 bp, Homo sapiens
aaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcag

SEQ ID NO: 2 - protein, 108 aa, Homo sapiens
MLLGQLSTLLCLLSGALPTGSGRPEPQSPRPQSWAAANQTWALGPGALPPLVPASALGSWKAFLGLQKARQLGMGRLQRGQDEVAAVTLPLNPQEVIQGMCKAVPFVQ

SEQ ID NO: 3 - DNA, 246 bp, Homo sapiens
gtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatga

SEQ ID NO: 4 - protein, 81 aa, Homo sapiens
VFSRPGCSAIRLRNHLCFGHCSSLYIPGSDPTPLVLCNSCMPARKRWAPVVLWCLTGSSASRRRVKISTMLIEGCHCSPKA

SEQ ID NO: 5 - DNA, 721 bp, Homo sapiens
aaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatga

SEQ ID NO: 6 - protein, 189 aa, Homo sapiens
MLLGQLSTLLCLLSGALPTGSGRPEPQSPRPQSWAAANQTWALGPGALPPLVPASALGSWKAFLGLQKARQLGMGRLQRGQDEVAAVTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKA

SEQ ID NO: 7 - protein, 86 aa, Homo sapiens
RPEPQSPRPQSWAAANQTWALGPGALPPLVPASALGSWKAFLGLQKARQLGMGRLQRGQDEVAAVTLPLNPQEVIQGMCKAVPFVQ

SEQ ID NO: 8 - protein, 167 aa, Homo sapiens
RPEPQSPRPQSWAAANQTWALGPGALPPLVPASALGSWKAFLGLQKARQLGMGRLQRGQDEVAAVTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKA

SEQ ID NO: 9 - DNA, 504 bp, Homo sapiens
aggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatga

SEQ ID NO: 10 - DNA, 258 bp, Homo sapiens
aggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcag

SEQ ID NO: 11 - DNA, 570 bp, Homo sapiens
atgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatga

SEQ ID NO: 12 - DNA, 324 bp, Homo sapiens
atgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcag

SEQ ID NO: 13 - DNA, 719 bp, Homo sapiens
gtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttctctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatgaactgagcatctggatgggtgcacggagacacgcaccttggagaaatgaggggagatggaccaagaaagacgtggacctggatgatgt

SEQ ID NO: 14 - protein, 187 aa, Homo sapiens
MLLGQLSTLLCLLSGALPTGSGRPEPQSPRPQSWAAANQTWALGPGALPPLVPASALGSWKAFLGLQKARQLGMGRLQRGQDEVAAVTLPLNPQEVIQGMCKAVPFVLSRPGCSAIRLRNHLCFGHCSSLYIPGSDPTPLVLCNSCMPARKRWAPVVLWCLTGSSASRRRVKISTMLIEGCHCSPKA

SEQ ID NO: 15 - DNA, 561 bp, Homo sapiens
atgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttctctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagca

SEQ ID NO: 16 - DNA, 1275 bp, Homo sapiens
atggctacaggctcccggacgtccctgctcctggcttttggcctgctctgcctgccctggcttcaagagggatccgccaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 17 - protein, 399 aa, Homo sapiens
RPEPQSPRPQSWAAANQTWALGPGALPPLVPASALGSWKAFLGLQKARQLGMGRLQRGQDEVAAVTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 987 bp, Homo sapiens
atggctacaggctcccggacgtccctgctcctggcttttggcctgctctgcctgccctggcttcaagagggatccgccaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - protein, 303 aa, Homo sapiens
RPEPQSPRPQSWAAANQTWALGPGALPPLVPASALGSWKAFLGLQKARQLGMGRLQRGQDEVAAVTLPLNPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1062 bp, Homo sapiens
atggctacaggctcccggacgtccctgctcctggcttttggcctgctctgcctgccctggcttcaagagggatccgcccaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - protein, 328 aa, Homo sapiens
QEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 570 bp, Homo sapiens
atggctacaggctcccggacgtccctgctcctggcttttggcctgctctgcctgccctggcttcaagagggatccgccgcggccgcgaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctgcggccgcggcccccgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgt

SEQ ID NO: 23 - protein, 161 aa, Homo sapiens [sequence omitted]

SEQ ID NO: 24 - DNA, 744 bp, Homo sapiens
atggctacaggctcccggacgtccctgctcctggcttttggcctgctctgcctgccctggcttcaagagggatccgccgcggccgcgaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctgcggccgcgtccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagcccatcccgactcccggggccctcggacaccccgatcctcccacaa

SEQ ID NO: 25 - protein, 219 aa, Homo sapiens [sequence omitted]

SEQ ID NO: 26 - DNA, 525 bp, Homo sapiens
atggctcaccatcaccatcaccataggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagca

SEQ ID NO: 27 - protein, 175 aa, Homo sapiens
MHHHHHHARPEPQSPRPQSWAAANQTWALGPGALPPLVPASALGSWKAFLGLQKARQLGMGRLQRGQDEVAAVTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKA

SEQ ID NO: 28 - DNA, 20 bp, Artificial Sequence
  INSP002-CP1 Primer
ctcagccatacgcctccgaa

SEQ ID NO: 29 - DNA, 20 bp, Artificial Sequence
  INSP002-CP2 Primer
gctgagctgccagtgagaca

SEQ ID NO: 30 - DNA, 25 bp, Artificial Sequence
  INSP002V-5′-F Primer
acctggaaggaagcgactgcactga

SEQ ID NO: 31 - DNA, 37 bp, Artificial Sequence
  INSP002V-5′-R Primer
gcagccgggccgggagagaacgaagggcacagcctta

SEQ ID NO: 32 - DNA, 37 bp, Artificial Sequence
  INSP002V-3′-F Primer
aggctgtgcccttcgttctctcccggcccggctgctc

SEQ ID NO: 33 - DNA, 25 bp, Artificial Sequence
  INSP002V-3′-R Primer
actccaggacgggcactgtgtctac

SEQ ID NO: 34 - DNA, 22 bp, Artificial Sequence
  INSPO02V-5′nest-F Primer
gtcgactgctagtgaccttgag

SEQ ID NO: 35 - DNA, 22 bp, Artificial Sequence
  INSPO02V-3′nest-R Primer
acatcatccaggtccacgtctt

SEQ ID NO: 36 - DNA, 18 bp, Artificial Sequence
  SP6 Primer
atttaggtgacactatag

SEQ ID NO: 37 - DNA, 20 bp, Artificial Sequence
  T7 Primer
taatacgactcactataggg

SEQ ID NO: 38 - DNA, 20 bp, Artificial Sequence
  T3 Primer
attaaccctcactaaaggga

SEQ ID NO: 39 - DNA, 18 bp, Artificial Sequence
  M13F
tgtaaaacgacggccagt

SEQ ID NO: 40 - DNA, 20 bp, Artificial Sequence
  pEAK12-F Primer
gccagcttggcacttgatgt

SEQ ID NO: 41 - DNA, 20 bp, Artificial Sequence
  pEAK12-R Primer
gatggaggtggacgtgtcag

SEQ ID NO: 42 - DNA, 37 bp, Artificial Sequence
  INSP002V-EX1 Primer
aagcaggcttcgccaccatgctccttggccagctatc

SEQ ID NO: 43 - DNA, 35 bp, Artificial Sequence
  INSP002V-EX2 Primer
gtgatggtgatggtgtgcttttgggctgcagtgac

SEQ ID NO: 44 - DNA, 37 bp, Artificial Sequence
  GCP Forward Primer
ggggacaagtttgtacaaaaaagcaggcttcgccacc

SEQ ID NO: 45 - DNA, 51 bp, Artificial Sequence
  GCP Reverse Primer
ggggaccactttgtacaagaaagctgggtttcaatggtgatggtgatggtg

SEQ ID NO: 46 - DNA, 20 bp, Artificial Sequence
  INSP002-FL-F Primer
gatgctccttggccagctat

SEQ ID NO: 47 - DNA, 20 bp, Artificial Sequence
  INSP002-FL-R Primer
ccatccacgatgctcagttc

SEQ ID NO: 48 - protein, 13 aa, Artificial Sequence
  amino acid linker
EFGAGLVLGGQFM

SEQ ID NO: 49 - DNA, 50 bp, Artificial Sequence
  Nco-6H-002-Final F Forward Primer
gaattcccatggctcaccatcaccatcaccataggcctgaaccccagtct

SEQ ID NO: 50 - DNA, 31 bp, Artificial Sequence
  002-XhoI-R Reverse Primer
gaattcctcgagctatgcttttgggctgcag

SEQ ID NO: 51 - DNA, 33 bp, Artificial Sequence
  Nde-002-F Forward Primer
gggaaattccatatgaggcctgaaccccagtct

SEQ ID NO: 52 - DNA, 31 bp, Artificial Sequence; identical to SEQ ID NO: 50
  002-XhoI-R Reverse Primer

SEQ ID NO: 53 - protein, 102 aa, Homo sapiens
VTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPK

SEQ ID NO: 54 - protein, 97 aa, Homo sapiens
VILPIKSHEVHWETCRTVPFSQTITHEGCEKVVVQNNLCFGKCGSVHFPGAAQHSHTSCSHCLPAKFTTMHLPLNCTELSSVIKVVMLVEECQCKVK

SEQ ID NO: 55 - DNA, 778 bp, Homo sapiens
  exon (152)..(718)
aaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatgaactgagcatcgtggatgggtgcacggagacacgcaccttggagaaatgaggggagat

SEQ ID NO: 56 - DNA, 159 bp, Homo sapiens
  exon (4)..(159)
ctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagc

SEQ ID NO: 57 - DNA, 720 bp, Homo sapiens
cagaaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagc

SEQ ID NO: 58 - DNA, 159 bp, Homo sapiens; identical to SEQ ID NO: 56

SEQ ID NO: 59 - protein, 53 aa, Homo sapiens
SAIRLRNHLCFGHCSSLYIPGSDPTPLVLCNSCMPARKRWAPVVLWCLTGSSA

SEQ ID NO: 60 - DNA, 1756 bp, Homo sapiens
  exon (197)..(595)
cggcacgagggggagacctggaaggaagcgactgcactgatccagaaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcagacacgggagtctcgctatgttgcccaagctagtcttgagcttctggcctcaagcaatcctcccacctcagcctcctgagttctaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatgaactgagcatctggatgggtgcacggagacacgcaccttggagaaatgaggggagatggaccaagaaagacgtggacctggatgatgtactctgggtcaagagaccagggatgcagggttaggcagacaggtccccagagtcctcaccctgctccccagacagtagacacagtgcccgtcctggagttgcaccactgatagtcacagcacacaatgattgacaactcacttttttttttttttttgagatggagtctcgctctgtcgcccaggctggagtgcagtggcgcaatctcagctcactgcaagctccacctcccgggtttatgccattctcctgtctcagcctcccgagtagctgggactacaggcacccgccaacacgcccggstaattttttgtatttttagtaaagacagggtttcaccgtgttagccaggatggtctctatctcctgacctcgtgatctgcctgccttggccttattattttttttttaaggacagagtctctctctgtcacccaggctggagtgcaatggcgcgatcttggctcactgtaacttccacttgccaggctcaagcagttctcctgcctcagcctcctgagtagctgggactacaggcacccgccaccatgcccagctaatttttgtatttttagtagagacagagtttcaccatattagcctggctggtctcaaactcctggcctcaggtgatctgcccacctcggcctcccaaagtgctgggatcaaatccactgttaatcattaggctgaactgtctcttatagaatgaggtcaaagacactcccagttgcagggagggtagatggccccacccagaccgagagacacagtgatgacctcagcctagggacmccaaaaaaaaaaaaaaaaaaaaa

SEQ ID NO: 61 - DNA, 724 bp, Homo sapiens
cagaaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatga

SEQ ID NO: 62 - DNA, 1746 bp, Homo sapiens
gggagacctggaaggaagcgactgcactgatccagaaatgcctcccaggctatccaggaggggccaagagattaaaagcaggttcagaaggctcagatgccactcaccagacagcagggtcgactgctagtgaccttgagcccagtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcagacacgggagtctcgctatgttgcccaagctagtcttgagcttctggcctcaagcaatcctcccacctcagcctcctgagttctaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatgaactgagcatctggatgggtgcacggagacacgcaccttggagaaatgaggggagatggaccaagaaagacgtggacctggatgatgtactctgggtcaagagaccagggatgcagggttaggcagacaggtccccagagtcctcaccctgctccccagacagtagacacagtgcccgtcctggagttgcaccactgatagtcacagcacacaatgattgacaactcacttttttttttttttttgagatggagtctcgctctgtcgcccaggctggagtgcagtggcgcaatctcagctcactgcaagctccacctcccgggtttatgccattctcctgtctcagcctcccgagtagctgggactacaggcacccgccaacacgcccggctaattttttgtatttttagtaaagacagggtttcaccgtgttagccaggatggtctctatctcctgacctcgtgatctgcctgccttggccttattattttttttttaaggacagagtctctctctgtcacccaggctggagtgcaatggcgcgatcttggctcactgtaacttccacttgccaggctcaagcagttctcctgcctcagcctcctgagtagctgggactacaggcacccgccaccatgcccagctaatttttgtatttttagtagagacagagtttcaccatattagcctggctggtctcaaactcctggcctcaggtgatctgcccacctcggcctcccaaagtgctgggatcaaatccactgttaatcattaggctgaactgtctcttatagaatgaggtcaaagacactcccagttgcagggagggtagatggccccacccagaccgagagacacagtgatgacctcagcctagggacaccaaaaaaaaaaaaaaaaaaaaa

SEQ ID NO: 63 - protein, 133 aa, Homo sapiens
MLLGQLSTLLCLLSGALPTGSGRPEPQSPRPQSWAAANQTWALGPGALPPLVPASALGSWKAFLGLQKARQLGMGRLQRGQDEVAAVTLPLNPQEVIQGMCKAVPFVQTRESRYVAQASLELLASSNPPTSAS

SEQ ID NO: 64 - protein, 189 aa, Homo sapiens; identical to SEQ ID NO: 6

SEQ ID NO: 65 - DNA, 724 bp, Homo sapiens; identical to SEQ ID NO: 61

SEQ ID NO: 66 - protein, 187 aa, Homo sapiens; identical to SEQ ID NO: 14

SEQ ID NO: 67 - protein, 189 aa, Homo sapiens; identical to SEQ ID NO: 6

SEQ ID NO: 68 - DNA, 589 bp, Homo sapiens
  exon (2)..(568)
gatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatgaactgagcatcgtggatgg